(19) United States
(12) Patent Application Publication
(10) Pub. No.: US 2005/0079496 A1
(43) Pub. Date: Apr. 14, 2005

(54) Methods for diagnosing and treating neoplasias using nf-at transcriptions factors

(76) Inventors: Edgar Serfling, Oberpleichfeld (DE); Sergey Chuvpilo, Estenfeld (DE); Andris Avots, Wuerzburg (DE)

Correspondence Address: FOLEY AND LARDNER;SUITE 500, 3000 K STREET NW, WASHINGTON, DC, 20007, US

(21) Appl. No.: 10/486,970

(22) PCT Filed: May 12, 2002
(86) PCT No.: PCT/EP02/09012

(30) Foreign Application Priority Data
Aug. 17, 2001 (EP) 0119961.9

(51) Int. Cl. C12Q 1/68; G01N 33/574

(52) U.S. Cl. 435/6; 435/7.23

(57) ABSTRACT

Described is a method for diagnosing a neoplasia by determining whether the level of one or more NF-AT transcription factor(s) is reduced. Moreover, methods for screening compounds which activate an NF-AT or which mimic the function of NF-AT are disclosed. Also disclosed are methods for the prevention or treatment of neoplasias by increasing the activity of an NF-AT.

The present invention relates to a method for diagnosing a neoplasia by determining whether the expression and/or activity of one or more NF-AT transcription factor(s) is reduced or missing. Moreover, the present invention relates to methods for screening compounds which activate an NF-AT. Furthermore, the present invention relates to methods for preventing or treating neoplasias by increasing the activity of an NF-AT.

The transformation and malignant growth of lymphomas and other tumor cells is a complex process which varies not only between various tumor samples but also very often between tumors which are supposed to belong to the same type of tumors. Therefore, there is a need for molecular markers and techniques that can be used to define the phenotype of tumor cells, i.e. to diagnose the type of tumor, which in turn allows to develop an appropriate tumor therapy. There is also still a need for efficient means for preventing or treating neoplasias.

Thus, the technical problem underlying the present invention is to provide means and methods for diagnosing and preventing and/or treating neoplasias, in particular lymphomas such as T cell lymphomas or other neoplasias of the hematopoietic system.

This problem has been solved by the provision of the embodiments as characterized in the claims.

Accordingly, in a first aspect the present invention relates to a diagnostic method comprising the step of determining in a sample taken from a patient the expression level and/or activity of one or more NF-AT transcription factor(s) and wherein a decrease or loss in the expression and/or activity of the NF-AT transcription factor(s) is indicative for the occurrence of a neoplasia.

It has been surprisingly found that inactivation of NF-AT transcription factors correlates with severe defects in apoptosis of lymphoid cells and first signs of cancerogenesis. It has, moreover, been found that an integration sequence for the T-cell lymphoma virus SL3-3 is present in an NF-AT promoter and that insertion of T-cell lymphoma virus SL 3-3 into the murine NF-ATc1 promoter leads to the concomitant total loss of NF-ATc1 expression. Moreover, it was found that in about 50% of more than 20 tested T-cell lymphomas the NF-ATc1 gene was inactivated, in particular in Hodgkin lymphomas and anaplastic large cell lymphomas (ALCLs). Finally, the promoters of NF-AT transcription factor genes were found to contain a high number of CpG residues, which are targets of methylation in eukaryotic cells, and methylation appears to be increased in tumor cells leading to inactivation of the genes. All these findings indicate that the NF-AT factors constitute tumor suppressors whose activity plays an important role, e.g., in the generation of T cell lymphomas, but probably also of other neoplasias. They may affect all cell types in which NF-AT factors are found to be expressed, such as all kinds of lymphoid cells (T, B, NK cells), further hematopoietic cells, muscle cells, chondrocytes, bone cells, blood vessel cells etc.

The sample used in the method according to the invention which is used for determining the expression level of one or more NF-AT transcription factor(s) can be any sample suitable for such a determination. Such samples include organ or tissue culture derived fluids, body fluids, such as blood, or biopsy samples or other sources of tumor cells. Also included in the term “sample” are derivatives and fractions of the above-mentioned fluids, tissues or cells. The number of cells in a sample is preferably at least about 10³, more preferably at least 10⁴, still more preferably at least 10⁵ and most preferably more than 10⁶. The cells may be dissociated in the case of solid tissues, or tissue sections may be analyzed. Alternatively, a lysate of the cells may be prepared.

Preferably the sample is a biopsy or another cell sample of the tumor. Most preferably the sample is a biopsy of a lymph node or from another peripheral lymphoid organ, such as spleen, tonsils, etc., or another cell sample of the tumor.

In a preferred embodiment, in particular where the tumor has metastasized, the sample used in the method according to the invention is blood or is derived from blood. More preferably the sample comprises lymphocytes, even more preferably T-lymphocytes and most preferably peripheral T-lymphocytes. In special cases an enrichment of T-lymphocyte-like cells can be performed.

The term “NF-AT transcription factor” in the context of the present invention means a “Nuclear Factor of Activated T cell” transcription factor. Such a factor was originally described as a putative transcription factor in nuclear protein extracts from activated Jurkat T cells binding to the human IL-2 promoter (Shaw et al., Science 241 (1988), 202-205). Subsequently, this factor was identified as a target for the immunosuppressants cyclosporin A (CsA) and FK506 (Brabletz et al., Nucl. Acids Res. 19 (1991), 61-67; Emmel et al., Science 246 (1989), 1617-1620; Mattila et al., EMBO J. 9 (1990), 4425-4433; Randak et al., EMBO J. 9 (1990), 2529-2536), which are efficient inhibitors of T cell activation (Liu et.al., J. Immunol. 162 (1999), 4755-4761).

The term “NF-AT transcription factor” refers to a polypeptide which is able to transactivate transcription from a promoter which comprises an NF-AT binding site which is characterized by the core sequence T/AGGAAA. Such a transactivation can be tested for, e.g., by linking a promoter comprising such a binding site to a reporter gene (e.g. the luciferase or the CAT gene), contacting such a construct with the polypeptide to be tested (e.g. in an in vitro system or in a transient transfection assay) and determining whether there occurs transactivation.

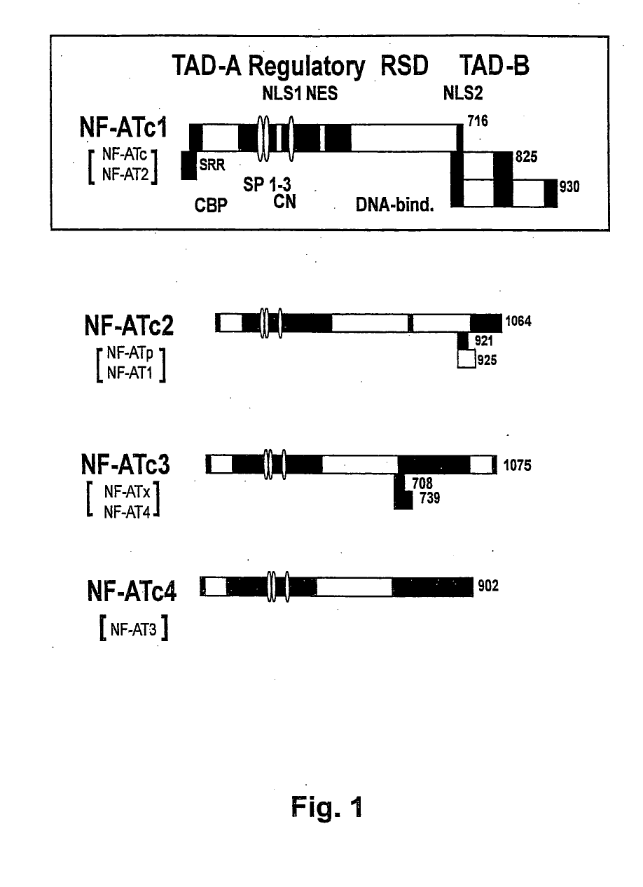

Today four closely related members of the NF-AT family have been cloned and characterized in detail. These four NF-AT proteins, designated as NF-ATc1 to NF-ATc4 (or NF-AT1 to 4; see FIG. 1 for other names) share a DNA binding domain of approximately 300 amino acid residues which is highly conserved between the different members of this family (the sequence homology ranges from 68 to 73% between the various NF-AT proteins). This DNA binding domain is often referred to as RSD (Rel Similarity Domain; see FIG. 1) due to its similarity to the DNA binding (Rel) domain of Rel/NF-κB factors, which is reflected in a very similar architecture although the similarity on the amino acid level is only 17 to 19%.

Thus, in a preferred embodiment the term “NF-AT transcription factor” refers to a transcription factor comprising an RSD domain.

Moreover, the term “NF-AT transcription factor” means a protein which, in addition to the RSD domain, preferably also contains a regulatory domain in front of the RSD. Preferably, this regulatory domain harbors several to numerous phosphorylation sites which are organized in a conserved serine-rich domain (SRD) and three so-called “SP” motifs. These motifs are repeating serine/proline/X/X motifs which are assumed to play a role in nuclear transport. These sites are substrates for several Ser/Thr protein kinases and for the protein phosphatase calcineurin which binds to this region.

Furthermore, an “NF-AT transcription factor” contains preferably at least one TAD (transactivating domain) near its N-terminus.

Preferably, also a nuclear localization signal (NLS) and/or a nuclear export signal (NES) are present in the regulatory region (Beals et al., Genes Dev. 11 (1997), 824-834; Klemm et al., Curr. Biol. 7 (1997), 638-644).

NF-AT transcription factors are also characterized by the property that they are bound-by calcineurin. Calcineurin binds to and de-phosphorylates NF-AT proteins thereby inducing their transport into the nucleus (Rao et al., Ann. Rev. Immunol. 15 (1997), 707-747; Serfling et al., Biochim. Biophys. Acta 1498 (2000), 1-18). Calcineurin consists of two polypeptides, a large, catalytic subunit A of 59 kD and a subunit B of 19 kD, and exerts a number of divergent cellular functions by binding to IP3, ryanodine and TGF-β receptors (Cameron et al., J. Biol. Chem. 272 (1997), 27582-27588; Cameron et al., Cell 83 (1995), 463-472; Wang et al., Cell 86 (1996), 435-444). Calcineurin interacts directly with several portions of the regulatory region of NF-AT factors (Luo et al., Proc. Natl. Acad. Sci. USA 93 (1996), 8907-8912; Masuda et al., Mol. Cell. Biol. 17 (1997), 2066-2075; Wesselborg et al., J. Biol. Chem. 271 (1996), 1274-1277). For NF-ATc3 two regions have been mapped spanning amino acids 25 to 143 and 321 to 406 (Liu et al., J. Immunol. 162 (1999), 4755-4761). The more N terminal peptide contains a version of the SPRIEIT motif which spans the amino acids 110-116 in murine and human NF-ATc2 (and 117-123 in NF-AT2) and was found to be essential for the effective recognition and dephosphorylation of NF-ATc2 by calcineurin (Aramburu et al., Mol. Cell 1 (1998), 627-637). By using an affinity-driven peptide selection procedure a modified form of SPRIEIT has been synthesized, designated as VIVIT, which was approximately 25 fold more effective in inhibiting the binding of calcineurin to NF-ATc2 (Aramburu et al., Science 285 (1999), 2129-2133). Expression of a chimeric GST-VIVIT protein efficiently inhibited the calcineurin-dependent nuclear translocation of NF-ATc2 and the activation of an NF-AT/AP-1-driven reporter gene but did not impair calcineurin activity. Thus, VIVIT enables to distinguish between NF-AT and calcineurin function and, therefore, to determine NF-AT- (and not calcineurin-) specific target genes (Aramburu et al. (1999); loc. cit.).

The property of an NF-AT to bind calcineurin can, e.g., be determined by incubation with low doses (10-100 ng/ml) of cyclosporin A which is able to block any NF-AT activity by binding to calcineurin (in a complex with a cyclophilin), inhibiting its activity and, thereby, inhibiting the nuclear translocation of the NF-ATs.

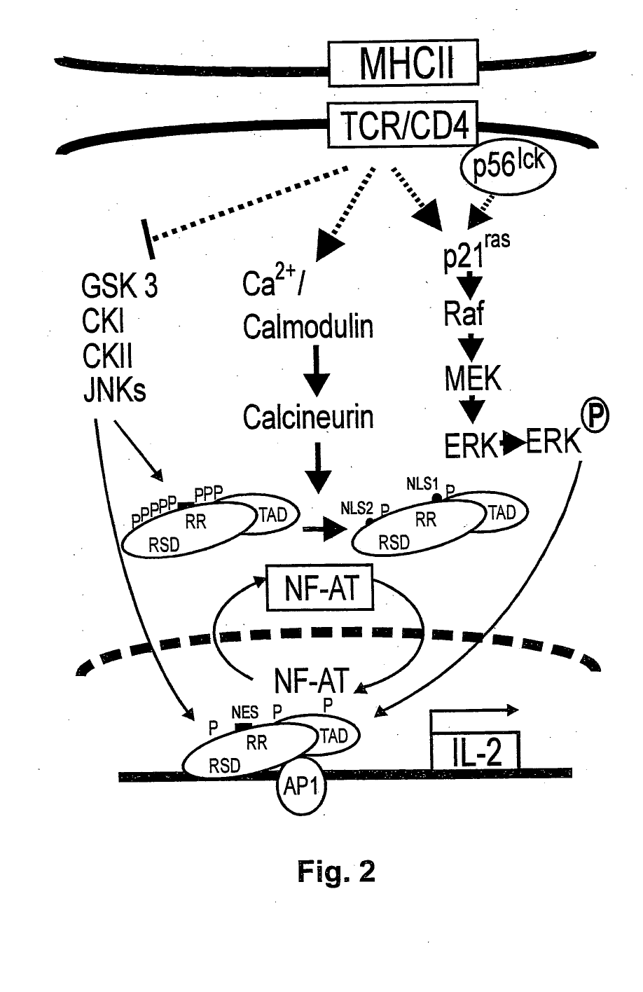

NF-AT transcription factors furthermore show the characteristic that upon dephosphorylation of several phosphoacceptor sites the NF-AT/calcineurin complexes are translocated into the nucleus (Shibasaki et al., Nature 382 (1996), 370-373) where they stimulate transcription. This needs persistent high levels of free cellular Ca⁺⁺ providing sustained Ca⁺⁺ signals (Timmerman et al., Nature 383 (1996), 837-840) since, otherwise, the active NF-AT/calcineurin complexes dissociate, and the NF-AT proteins (and calcineurin) are rapidly exported to the cytosol (see FIG. 2).

NF-AT transcription factors moreover show the characteristic that they can be phosphorylated by several Ser/Thr protein kinases, such as glycogen synthase kinase-3 (GSK-3), casein kinase lα (CKlα) and several others (see Serfling et al., Biochim. Biophys. Acta 1498 (2000), 1-18, for a discussion). This phosphorylation counteracts the activity of calcineurin. NF-AT transcription factors are furthermore characterized in that the efficient transcriptional activation of NF-AT factors in T cells needs at least two signals which are provided by activation of the T cell receptor (TCR). These TCR-mediated signals lead to (1.) a rise in intracellular free Ca⁺⁺ and calcineurin activation, and (2.) the stimulation of several protein tyrosine kinases, e.g. p56^{lck}, and p21^{ras} and other small GTP binding proteins which activate a number of Ser/Thr protein kinase cascades (FIG. 2). While the activation of calcineurin mediates predominantly the nuclear translocation of NF-AT factors (Liu, Immunol. Today 14 (1993), 290-295), activation of classical Ras/Raf/Erk and further protein kinase cascades controls the transcriptional activation of NF-ATs (Avots et al., Immunity 10 (1999), 515-524) and the induction of AP-1 (Treisman, Curr. Opin. Cell Biol. 8 (1996), 205-215) which facilitates the binding of NF-ATs to composite NF-AT+AP-1 elements which are typical for promoters active in T cells (see FIG. 2).

Another characteristic property of NF-ATs is their sensitivity against low doses (such as 10 to 100 ng/ml) of cyclosporin A (CsA). Cyclosporin specifically inhibits the activity of calcineurin and, thus, the nuclear translocation of the NF-ATs.

In a preferred embodiment the term “NF-AT transcription factor” means an NF-AT transcription factor selected from the group consisting of:
(i) NF-ATc1;
(ii) NF-ATc2;
(iii) NF-ATc3; and
(iv) NF-ATc4.

In this context, the term “NF-ATc1” refers to a protein which can be classified as NF-ATc1 due to the following characteristics:
(a) it is highly expressed after T-cell receptor induction in peripheral T cells (Northrop et al., Nature 369 (1994), 497-502);
(b) it is synthesized in three major isoforms in peripheral T cells which differ substantially in their C terminal portions (Chuvpilo et al., Immunity 10 (1999), 261-269); and
(c) its inactivation leads to embryonic lethality due to defects in differentiation of cardiac valves and septa (Ranger et al., Nature 392 (1998), 186-190; de la Pompa et al., Nature 392 (1998), 182-186).

More preferably the term “NF-ATc1” refers to an NF-AT transcription factor which is encoded by a nucleic acid molecule selected from the group consisting of:
(a) nucleic acid molecules which encode a protein comprising the amino acid sequence as depicted in SEQ ID NO:2 or SEQ ID NO:10;
(b) nucleic acid molecules comprising the coding region of the nucleotide sequence as depicted in SEQ ID NO:1 or SEQ ID NO:9;
(c) nucleic acid molecules the complementary strand of which hybridizes to a nucleic acid molecule of (a) or (b); and
(d) nucleic acid molecules the sequence of which differs from the sequence of a nucleic acid molecule of (c) due to the degeneracy of the genetic code.

The cDNA sequences shown in SEQ ID NO:1 and 9, respectively, relate to different isoforms of NF-ATc1, i.e. isoforms NF-ATc1/C (SEQ ID NO:1) and NF-ATc1/A (SEQ ID NO:9). The isoform C is longer than the isoform A. In contrast to the isoform A it contains an additional C-terminal peptide of 246 amino acid residues.

In this context, the term “NF-ATc2” refers to a protein which can be classified as NF-ATc2 due to the following characteristics:
(a) it is highly expressed in peripheral T cells (in contrast to NF-ATc1 it is constitutively expressed); and
(b) its inactivation leads to defects in T cell apoptosis and in chondrocyte differentiation (Ranger et al., J. Exp. Med. 191 (2000), 9-22), but no defects in the development of embryonic heart can be observed.

More preferably the term “NF-ATc2” refers to an NF-AT transcription factor which is encoded by a nucleic acid molecule selected from the group consisting of:
(a) nucleic acid molecules which encode a protein comprising the amino acid sequence as depicted in SEQ ID NO:4;
(b) nucleic acid molecules comprising the coding region of the nucleotide sequence as depicted in SEQ ID NO:3;
(c) nucleic acid molecules the complementary strand of which hybridizes to a nucleic acid molecule of (a) or (b); and
(d) nucleic acid molecules the sequence of which differs from the sequence of a nucleic acid molecule of (c) due to the degeneracy of the genetic code.

In this context, the term “NF-ATc3” refers to a protein which can be classified as NF-ATc3 due to the following characteristics:
(a) it is highly expressed in double positive thymoctes;
(b) it is a relatively weak transactivator of lymphokine genes;
(c) its inactivation leads to defects in thymocyte development (Oukka et al., Immunity 9 (1998), 295-304); and
(d) it controls blood vessel formation along with NF-ATc4 (Graef et al., Cell 105 (2001), 863-875).

More preferably the term “NF-ATc3” refers to an NF-AT transcription factor which is encoded by a nucleic acid molecule selected from the group consisting of:
(a) nucleic acid molecules which encode a protein comprising the amino acid sequence as depicted in SEQ ID NO:6;
(b) nucleic acid molecules comprising the coding region of the nucleotide sequence as depicted in SEQ ID NO:5;
(c) nucleic acid molecules the complementary strand of which hybridizes to a nucleic acid molecule of (a) or (b); and
(d) nucleic acid molecules the sequence of which differs from the sequence of a nucleic acid molecule of (c) due to the degeneracy of the genetic code.

In this context, the term “NF-ATc4” refers to a protein which can be classified as NF-ATc4 due to the following characteristics:
(a) it is not or very weakly expressed in lymphoid cells; and
(b) in mice deficient for NF-ATc3 and c4 the blood vessel formation is disturbed, the murine embryos die before day 11 of gestation (Graef et al., Cell, 105 (2001), 863-875).

More preferably the term “NF-ATc4” refers to an NF-AT transcription factor which is encoded by a nucleic acid molecule selected from the group consisting of:
(a) nucleic acid molecules which encode a protein comprising the amino acid sequence as depicted in SEQ ID NO:8;
(b) nucleic acid molecules comprising the coding region of the nucleotide sequence as depicted in SEQ ID NO:7;
(c) nucleic acid molecules the complementary strand of which hybridizes to a nucleic acid molecule of (a) or (b); and
(d) nucleic acid molecules the sequence of which differs from the sequence of a nucleic acid molecule of (c) due to the degeneracy of the genetic code.

Within the present invention the term “hybridization” means hybridization under conventional hybridization conditions, preferably under stringent conditions, as for instance described in Sambrook and Russell, Molecular Cloning, A Laboratory Manual, 3^{rd} edition (2001) Cold Spring Harbour Laboratory Press, Cold Spring Harbour, N.Y. Within an especially preferred meaning the term “hybridization” means that hybridization occurs under the following conditions:

| | |
|---|---|
| Hybridization | 2 × SSC; 10 × Denhardt solution (Ficoll 400 + PEG + |
| buffer: | BSA; ratio 1:1:1); 0.1% SDS; 5 mM EDTA; 50 mM |
| | Na₂HPO₄; 250 μg/ml of herring sperm DNA; 50 |
| | μg/ml of tRNA; or |
| | 0.25 M of sodium phosphate buffer, pH 7.2; |
| | 1 mM EDTA |
| | 7% SDS |
| Hybridization | 65° C. |
| temperature | |
| T = | |
| Washing buffer: | 0.1 × SSC; 0.1% SDS |
| Washing | 65° C. |
| temperature | |
| T = | |

Nucleic acid molecules which hybridize with a nucleic acid molecule of the invention can, in principle, encode an NF-AT protein from any organism expressing such proteins. Preferably they are from human origin.

Nucleic acid molecules which hybridize with a molecule of the invention can for instance be isolated from genomic libraries or cDNA libraries of any organism comprising such molecules. Alternatively, they can be prepared by genetic engineering or chemical synthesis.

Such nucleic acid molecules may be identified and isolated by using the above-mentioned nucleic acid molecules encoding NF-ATc1, NF-ATc2, NF-ATc3 and NF-ATc4, respectively, or parts of such molecules or reverse complements of such molecules, for instance by hybridization according to standard methods (see for instance Sambrook and Russell, 2001, Molecular Cloning. A Laboratory Manual, 3^{rd} edition, Cold Spring Harbor Laboratory Press, Cold Spring Harbor, N.Y.).

Nucleic acid molecules possessing the same or substantially the same nucleotide sequence as indicated in SEQ ID NOs: 1, 3, 5 or 7 or parts thereof can, for instance, be used as hybridization probes. The fragments used as hybridization probes can also be synthetic fragments which are prepared by usual synthesis techniques, and the sequence of which substantially coincides with that of a nucleic acid molecule according to the invention.

The molecules hybridizing with one of the above-mentioned nucleic acid molecules also comprise fragments, derivatives and allelic variants of the above-described nucleic acid molecules encoding an NF-AT protein, in particular an NF-ATc1, NF-ATc2, NF-ATc3 or NF-ATc4. Herein, fragments are understood to mean parts of the nucleic acid molecules which are long enough to encode one of the described proteins, preferably having the activity of an NF-AT. In this connection, the term derivative means that the sequences of these molecules differ from the sequence of an above-described nucleic acid molecule in one or more positions and show a high degree of homology to such a sequence. In this context, homology means a sequence identity of at least 40%, in particular an identity of at least 60%, preferably of at least 65%, more preferably of at least 70%, even more preferably of at least 80%, in particular of at least 85%, furthermore preferred of at least 90% and particularly preferred of at least 95%. Most preferably homology means a sequence identity of at least n%, wherein n is an integer between 40 and 100, i.e. 40≦n≦100. Deviations from the above-described nucleic acid molecules may have been produced, e.g., by deletion, substitution, insertion and/or recombination.

Preferably, the degree of homology is determined by comparing the respective sequence with the nucleotide sequence of the coding region of SEQ ID No: 1, 3, 5 or 7. When the sequences which are compared do not have the same length, the degree of homology preferably refers to the percentage of nucleotide residues in the shorter sequence which are identical to nucleotide residues in the longer sequence. The degree of homology can be determined conventionally using known computer programs such as the ClustalW program (Thompson et al., Nucleic Acids Research 22 (1994), 4673-4680) distributed by Julie Thompson (Thompson@EMBL-Heidelberg.DE) and Toby Gibson (Gibson@EMBL-Heidelberg.DE) at the European Molecular Biology Laboratory, Meyerhofstrasse 1, D 69117 Heidelberg, Germany. ClustalW can also be downloaded from several websites including IGBMC (Institut de Genetique et de Biologie Moléculaire et Cellulaire, B.P.163, 67404 lllkirch Cedex, France; ftp://ftp-igbmc.u-strasbg.fr/pub/) and EBI (ftp://ftp.ebi.ac.uk/pub/software/) and all sites with mirrors to the EBI (European Bioinformatics Institute, Wellcome Trust Genome Campus, Hinxton, Cambridge CB10 1SD, UK).

When using ClustalW program version 1.8 to determine whether a particular sequence is, for instance, 90% identical to a reference sequence according to the present invention, the settings are set in the following way for DNA sequence alignments:
KTUPLE=2, TOPDIAGS=4, PAIRGAP=5, DNAMATRIX:IUB, GAPOPEN=10, GAPEXT=5, MAXDIV=40, TRANSITIONS: unweighted.

For protein sequence alignments using ClustalW program version 1.8 the settings are the following: KTUPLE=1, TOPDIAG=5, WINDOW=5, PAIRGAP=3, GAPOPEN=10, GAPEXTEND=0.05, GAPDIST=8, MAXDIV=40, MATRIX=GONNET, ENDGAPS(OFF), NOPGAP, NOHGAP.

Furthermore, homology means preferably that the encoded protein displays a sequence identity of at least 40%, more preferably of at least 60%, even more preferably of at least 80%, in particular of at least 90% and particularly preferred of at least 95% to the amino acid sequence depicted under SEQ ID NO: 2, 4, 6 or 8. Most preferably homology means that there is a sequence identity of at least n%, wherein n is an integer between 40 and 100, i.e. 40≦n≦100.

Homology, moreover, means that there is a functional and/or structural equivalence between the corresponding nucleic acid molecules or proteins encoded thereby. Nucleic acid molecules which are homologous to one of the above-described molecules and represent derivatives of these molecules are generally variations of these molecules which represent modifications having the same biological function. They may be either naturally occurring variations, for instance sequences from other organisms, or mutations, and said mutations may have formed naturally or may have been produced by deliberate mutagenesis. Furthermore, the variations may be synthetically produced sequences. The allelic variants may, e.g., be naturally occurring variants or synthetically produced variants or variants produced by recombinant DNA techniques.

The proteins encoded by the different variants of one of the nucleic acid molecules of the invention possess certain characteristics they have in common. These include for biological activity, molecular weight, immunological reactivity, conformation, etc., and physical properties, such as for instance the migration behavior in gel electrophoreses, chromatographic behavior, sedimentation coefficients, solubility, spectroscopic properties, stability, pH optimum, temperature optimum etc.

One characteristic of the proteins encoded by one of the nucleic acid molecules of the invention is that they are NF-AT proteins (see above).

The term “variant” or “derivative” also embraces isoforms of NF-AT proteins. It is known that all NF-AT factors are synthesized in several isoforms which can differ both in their N- and C-terminal peptides (Chuvpilo et al., Immunity 10 (1999), 261-269; Imamura et al., J. Immunol. 161 (1998), 3455-3463; Luo et al., Mol. Cell. Biol. 16 (1996), 3955-3966; Lyakh et al., Mol. Cell. Biol. 17 (1997), 2475-2484; Park et al., .J. Biol. Chem. 271 (1996), 20914-20921; Sherman et al., J. Immunol. 162 (1999), 2820-2828). NF-ATc1, and in a similar way also NF-ATc2 and NF-ATc3, is synthesized in three major isoforms which differ markedly in the length of their C-termini. The C-terminal extra-peptides of the longest isoform NF-ATc1/C harbor a second, albeit weak TAD which shares approximately 30% sequence homology with the C-terminal sequences of NF-ATc2 (Chuvpilo et al., J. Immunol. 162 (1999), 7294-7301). The corresponding sequences in NF-ATc2 also harbor a TAD (Luo et al., J. Exp. Med. 184 (1996), 141-147).

The determination of the expression level and/or activity of one or more NF-AT transcription factor(s) in the sample taken from a patient can be carried out by methods well-known to the person skilled in the art. For example, the expression can be determined on the level of RNA synthesis, i.e. by determining the amount of RNA, or nucleic acids derived therefrom, coding for an NF-AT transcription factor, or on the protein level, i.e. by determining the amount or the activity of the NF-AT transcription factor present in the sample. Moreover, it is possible to determine loss of NF-AT gene expression on the level of DNA by determination of an LOH (loss of heterozygosity) or by methylation studies of, e.g., the promoter regions of NF-ATc1 and other NF-AT factors .

A multitude of techniques is available for determining the expression of a certain gene at the RNA level. Examples are in situ hybridization, Northern Blot analysis, Dot Blot analysis, PCR amplification, oligonucleotide ligation assays, binding to solid state arrays, microarray analysis etc.

It is possible to use either directly mRNA or total RNA derived from the sample for detecting the expression level of NF-ATs or alternatively, the RNA may be further reverse transcribed or amplified into cDNA. Amplification maybe carried out according to conventional methods, such as the polymerase chain reaction (PCR). This technique is, e.g., described in Sambrook and Russell (Molecular Cloning: A Laboratory Manual, CSH Press (2001 Chapter 8.1)

The RNA or amplification product can, for example, be fractionated by electrophoresis, e.g. capillary or gel electrophoresis, transferred to a suitable support, e.g. nitrocellulose, and further analyzed with regard to its amount.

In Northern Blot analysis, e.g., mRNA or total RNA is isolated from the sample according to known methods and is separated in a suitable gel. The RNA is subsequently transferred to a membrane, fixed and hybridized with a probe specific for the respective NF-AT transcription factor(s).

As indicated above, also amplification can be used to detect the presence of NF-AT transcription factor(s) encoding sequences in an RNA preparation derived from the sample. For this purpose, primers are used for a PCR amplification which will specifically bind to the respective NF-AT encoding sequence(s). The exact composition of the primer sequences is not critical as long as they allow detection of the desired sequence(s). Preferably, the primers are chosen in such a way that they hybridize under stringent conditions to the desired sequence(s). It is preferable to choose a pair of primers that will generate an amplification product of at least 50 nt, preferably of at least about 100 nt and most preferably of at least 200 nt. Algorithms for the selection of primer sequences are generally known and are available in commercial software packages. Amplification primers hybridize to complementary strands of DNA and will prime towards each other.

The sample nucleotide acid, e.g. the RNA or the amplification product, is then analyzed by one of a number of methods well-known in the art. The amount of NF-AT encoding molecules can, e.g., be determined by hybridization to an NF-AT-specific sequence making use, e.g., of Northern Blot, Dot Blot, microarray analysis etc.

The hybridization probe is chosen in such a way that it allows detection, preferably specific detection, of the desired sequence(s). More preferably, the hybridization probe is chosen so that it hybridizes under stringent conditions to the desired sequence(s). Most preferably, the hybridization probe is chosen in such a way that its sequence is at least 90%, in particular at least 95%, still more preferably at least 99% and most preferably 100% identical to a region of the sequence which is tested.

A hybridization probe should in general be at least 15 nt in length, more preferably at least 25 nt, even more preferably at least 50 nt, still more preferably at least 100 nt and most preferably at least 200 nt. The hybridization probe may be cDNA, RNA, a fragment of the afore-mentioned or a chemically synthesized nucleic acid molecule. It may also be an RNA/DNA-hybrid molecule and it may contain modifications.

For hybridization probes, it may be, e.g., desirable to use nucleic acid analogs, in order to improve the stability and binding affinity. The term “nucleic acid” shall be understood to encompass such analogs. A number of modifications have been described that alter the chemistry of the phosphodiester backbone, sugars or heterocyclic bases. Among useful—changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire phosphodiester backbone with a peptide linkage. Sugar modifications are also used to enhance stability and affinity. The a-anomer of deoxyribose may be used, where the base is inverted with respect to the natural b-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

The hybridization probe or the primer(s) used for amplification may also contain a detectable label. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine(ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may also be a two stage system, where the DNA is conjugated to biotin, haptens, etc. having a high affinity binding partner, e.g. avidin, specific antibodies, etc., where the binding partner is conjugated to a detectable label. In the case of amplification the label may be conjugated to one or both of the primers. Alternatively, the pool of nucleotides used in the amplification is labeled, so as to incorporate the label into the amplification product.

The hybridization probe or the amplification primer(s) may be chosen in such a way that they only detect one specific NF-AT transcription factor. In this case, their sequence has to be specific for this factor.

Alternatively, the probe or the primer(s) can be chosen so as to detect two or more NF-ATs. In this case, their sequence must be common to the sequences of the NF-ATs to be detected.

If it is intended to only test for expression levels of NF-ATs without distinguishing between the different types of NF-ATs, the probe or primer(s) have a sequence which is shared by all NF-AT mRNAs. An example for such a pan-NF-AT probe has the following degenerated sequence:
pan NFAT RSD direct primer: CAYCAYCGRGCCCAYTAYGARAC (SEQ ID NO:11)
pan NFAT RSD reversed primer: TCBTGRGCWGABCGCTGGGAG (SEQ ID NO:12)
   where Y=C,T; R=A,G; B=C,T,G; and W=A, T.

It is also possible to use less conserved regions of the NF-AT genes in order to generate probes or primers that distinguish NF-ATs from other transcription factor sequences. Such sequences include the 3′- and 5′-untranslated regions of the NF-AT cDNAs.

The determination of the amount of nucleic acid present in the sample which encodes NF-AT(s) may also be determined by making use of arrays onto which, e.g., oligonucleotide probes are immobilized and by determining hybridization patterns.

Such arrays and the corresponding technique are, e.g., described in U.S. Pat. No. 5,445,943 or in WO 95/35505. Also Hacia et al. (Nature Genetics 14 (1996), 441-447), Lockhart et al. (Nature Biotechnol. 14 (1996), 1675-1680) and DeRisi et al. (Nature Genetics 14 (1996), 457-460) describe such arrays.

As mentioned above, the expression level of NF-AT can also be determined by detecting the amount of NF-AT protein in the sample. In this regard, a multitude of techniques is available to the person skilled in the art which can generally all be used in the scope of the present invention. One example are assays which are based on a specific interaction of an NF-AT with a binding partner and detection of the formation of a complex between the NF-AT and the binding partner. “Binding partner” means a compound which binds to an NF-AT via chemical and/or physical means.

A typical example is an antibody which recognizes an NF-AT. Such antibodies can be monoclonal or polyclonal or they may be humanized. The term “antibody” also includes fragments of antibodies which still have the ability to recognize the NF-AT. The details of the preparation of antibodies and fragments thereof are well-known to the person skilled in the art.

Monoclonal antibodies for NF-ATc1 are, e.g., described in Northrop et al. (Nature 369 (1994), 497-502) and are distributed by ALEXIS Chemicals (Cat. No. 804-022-R100). Polyclonal antibodies raised against all the other members of the NF-AT family are produced and distributed by SANTA CRUZ Biotechnology, Inc. Alternatively, monoclonal or polyclonal antibodies are raised to human NF-ATs. The antibodies may be produced in accordance with conventional methods, e.g., immunization of a mammalian host, e.g. mouse, rat, guinea pig, cat, dog, etc., fusion of resulting splenocytes with a fusion partner for immortalization and screening for antibodies having the desired affinity to provide monoclonal antibodies having a particular specificity.

The antibodies may be labeled, e.g., with radioisotopes, enzymes, fluorescers, chemiluminescers, or other label which will allow for detection of complex formation between the labeled antibody and its complementary epitope. Generally the amount of bound NF-ATs detected will be compared to negative control samples from normal tissue or from known non-responsive carcinoma cells.

The antibodies can be used in different assays known to the person skilled in the art to determine the amount of NF-AT protein. These include but are not limited to affinity chromatography, ELISA, RIA, Western Blot analysis, immunofluorescent histochemistry, fluorescent microscopy, immunoelectron microscopy and other diagnostic methods.

If it is intended to determine only the amount of one specific NF-AT protein, an antibody will be used which determines specifically this protein, i.e. an epitope which is specific for this protein and which is not present in the other NF-AT proteins. If more than one type of NF-AT protein should be detected, an antibody is used which recognizes an epitope which is shared by the respective NF-AT proteins. Alternatively, a mixture of antibodies which are specific for one (or more) NF-ATs is used.

The amount of NF-AT protein can also be determined by other methods based on its interaction with a specific binding partner, in particular with the DNA motif which is recognized by the protein. The DNA motifs to which the NF-AT proteins bind are known and share the “core” motif T/AGGAAA (see compilation in Rao et al., Annual Rev. Immun. 15 (1997), 707-747 and Serfling et al., Biochem. Biophys. Acta 1498 (2000), 1-18). This interaction and the formation of a complex between the NF-AT protein and its DNA motif may be detected by methods known to the person skilled in the art. Generally, an oligonucleotide comprising the DNA motif is labeled and is used, e.g., in South Western analysis, gel shift assay (Electrophoretic Mobility Shift Assay (EMSA)), DNA footprints or the like.

Detection may utilize staining of cells or histological sections, performed in accordance with conventional methods. The antibodies or other specific binding partners of interest are added to the cell sample, and incubated for a period of time sufficient to allow binding to the protein/epitope, usually at least about 10 minutes. The binding partner may be labeled with radioisotopes, enzymes, fluorescers, chemiluminescers, or other labels for direct detection. Alternatively, a second stage binding partner, e.g. an antibody or reagent, is used to amplify the signal. Such reagents are well known in the art. For example, the primary binding partner, e.g. an antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

In a preferred embodiment the method for diagnosis depends on the in vitro detection of binding between a binding partner, such as an antibody, and NF-ATs in a lysate. Measuring the concentration of NF-AT binding in a sample or fraction thereof may be accomplished by a variety of specific assays. A conventional sandwich type assay may be used. For example, a sandwich assay may first attach on NF-AT specific binding partner, e.g. an antibody, to an insoluble surface or support. The particular manner of binding is not crucial so long as it is compatible with the reagents and overall methods of the invention. They may be bound to the plates covalently or non-covalently, preferably non-covalently.

The insoluble support may be any compositions to which the binding partner, in particular polypeptides, can be bound, which is readily separated from soluble material, and which is otherwise compatible with the overall method. The surface of such a support may be solid or porous and of any convenient shape. Examples of suitable insoluble supports to which the binding partner is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Patient sample lysates are then added to separately assailable supports (for example, separate wells of a microtiter plate) containing the binding partner. Preferably, a series of standards, containing known concentrations of NF-ATs is assayed in parallel with the samples or aliquots thereof to serve as controls. Preferably, each sample and standard will be added to multiple wells so that mean values can be obtained for each. The incubation time should be sufficient for binding, generally, from about 0.1 to 3 hr is sufficient. After incubation, the insoluble support is generally washed of non-bound components. Generally, a dilute non-ionic detergent medium at an appropriate pH, generally 7-8, is used as a wash medium. From one to six washes may be employed, with sufficient volume to thoroughly wash non-specifically bound proteins present in the sample.

After washing, a solution containing an antibody which recognizes NF-AT or another NF-AT specific binding partner is applied. This binding partner will bind NF-AT factors with sufficient specificity such that it can be distinguished from other components present. The second binding partner may be labeled to facilitate direct, or indirect quantification of binding. Examples of labels that permit direct measurement of the binding of the second partner include radiolabels, such as ³H or ¹²⁵I, fluorescers, dyes, beads, chemilumninescers, colloidal particles, and the like. Examples of labels that permit indirect measurement of binding include enzymes where the substrate may provide for a colored or fluorescent product. In a preferred embodiment, the second binding partner is labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The incubation time should be sufficient for the labeled ligand to bind available molecules. Generally, from about 0.1 to 3 hr is sufficient, usually 1 hr sufficing.

After the second binding step, the insoluble support is again washed free of non-specifically bound material, leaving the specific complex formed between NF-ATs and the specific binding partner. The signal produced by the bound conjugate is detected by conventional means. Where an enzyme conjugate is used, an appropriate enzyme substrate is provided so a detectable product is formed.

Other immunoassays are known in the art and may find use as diagnostics. Ouchterlony plates provide, e.g., a simple determination of antibody binding. Western blots may be performed on protein gels or protein spots on filters, using a detection system specific for NF-ATs as desired, conveniently using a labeling method as described for the sandwich assay.

In some cases, a competitive assay may be used. In addition to the patient sample, a competitor to NF-ATs is added to the reaction mix. The competitor and the NF-AT proteins compete for binding to the specific binding partner. Usually, the competitor molecule will be labeled and detected as previously described, where the amount of competitor binding will be proportional to the amount of NF-AT factors present. The concentration of competitor molecule will be from about 10 times the maximum anticipated NF-AT concentration to about equal concentration in order to make the most sensitive and linear range of detection.

It is particularly convenient in a clinical setting to perform the immunoassay in a self-contained apparatus. A number of such methods are known in the art. The apparatus will generally employ a continuous flow-path of a suitable filter or membrane, having at least three regions, a fluid transport region, a sample region, and a measuring region. The sample region is prevented from fluid transfer contact with the other portions of the flow path prior to receiving the sample. After the sample region receives the sample, it is brought into fluid transfer relationship with the other regions, and the fluid transfer region contacted with fluid to permit a reagent solution to pass through the sample region and into the measuring region. The measuring region may have bound to it a conjugate of an enzyme with an NF-AT protein-specific antibody.

Other assays for determining the amount and/or activity of NF-AT protein are based on a functional characteristic of an NF-AT protein, in particular on its property to direct transcription. Such functional tests can be carried out in in vitro transcription systems after isolating NF-AT factors or by using cloned cDNAs in transient transfection assays with NF-AT-site directed reporter genes (e.g. luciferase genes).

Moreover, there exists the possibility to determine on the DNA level whether or not an NF-AT gene is expressed. It is, for example, possible that methylation of the promoter region of an NF-AT gene leads to a suppression of transcription of this gene. Thus, determining the methylation state of an NF-AT gene promoter may allow to determine whether this NF-AT gene is transcribed. Methylation studies can be carried out by methods well known to the person skilled in the art. A further possibility is the detection of an LOH (loss of heterozygosity) by using microsatellite analyses. For this purpose microsatellite markers from the chromosomal loci of the NF-AT genes are used.

The level of expression and/or activity of NF-AT in the sample is compared to a control. The control may be, e.g., a sample from normal, i.e. not neoplastic tissue or body fluid, of the same patient or a sample from a patient known to be healthy, i.e. not having the neoplasia to be tested for.

The term “a decrease in the expression level and/or activity of the NF-AT transcription factor(s)” means a decrease of the level of expression and/or activity determined in the sample when compared to the control, preferably by at least 50%, more preferably by at lest 60%, even more preferably by at least 70%, still more preferably by at least 80% and most preferably by at least 100%, i.e. a complete loss of expression and/or activity.

The neoplasia to be diagnosed by the method according to the invention is preferably a neoplasia of a tissue in which an NF-AT factor is expressed. It is supposed that the reduction or loss of expression of already one individual NF-AT factor leads to tumor generation. Examples are all kinds of hematopoietic cells, including peripheral T- and B-lymphocytes and NK cells in which NF-ATc1 and NF-ATc2 are highly expressed, as well as thymocytes, in which NF-ATc3 is expressed, chondrocytes and osteocytes (in which NF-ATc2 is expressed), heart, muscle and blood vessel cells in which NF-ATc1, c3 and c4 are expressed. The generation of any possible neoplasia of these tissues/cells might be promoted by the reduction or loss of NF-AT expression.

In a preferred embodiment the neoplasia is a lymphoma, more preferably a T-cell lymphoma or a B-cell lymphoma. These can be in particular be diagnosed by a reduction or loss of expression/activity of NF-ATc1 and/or NF-ATc2 and/or of NF-ATc3.

In another preferred embodiment the neoplasia is a leukemia. These can in particular be diagnosed by a reduction or loss of expression/activity of NF-ATc1 and/or NF-ATc2 and/or of NF-ATc3.

In another preferred embodiment the neoplasia is a neoplasia of chondrocytes or osteocytes. These can in particular be diagnosed by a reduction or loss of expression/activity of NF-ATc2.

Neoplasias of non-lymphoid tissues, such as muscle tissue or blood vessel cells, can be diagnosed by a loss or reduction of expression/activity of NF-ATc3 and/or NF-ATc4.

More preferably the neoplasia to be diagnosed is a lymphoma, most preferably a T cell lymphoma and particularly preferred a peripheral T-cell lymphoma.

According to morphological and immunohistochemical criteria the two classes of T cell lymphomas, i.e. low-grade and high-grade malignant lymphomas, can be classified into a number of subclasses (T-CLL, T-PLL, “Lennert's Lymphoma” etc.). which show very often a quite different prognosis (Lennert and Feller, Histopathology of Non-Hodgkin's Lymphomas. Springer-Vlg. 1992). It may be possible that according to molecular criteria, e.g. the expression levels of NF-AT factors, these lymphomas can be classified according to their defects at the molecular level, and an appropriate therapy can be applied. One example is the classification of diffuse large B-cell Lymphoma's (DLBCL) which could be classified according to gene expression data into two types, i.e. in Germinal Center B-like and activated B cell like DLBCLs (Alizadeh et al., Nature 403 (2000), 503-511). Whereas the former show a favorable diagnosis patients suffering from activated B cell DLBCL have a poor diagnosis and need a more aggressive therapy.

The present invention also relates to a diagnostic composition comprising one or more antibodies recognising one or more NF-AT factors or a nucleic acid molecule specifically hybridizing with at least one nucleotide sequence encoding an NF-AT protein or with a promoter sequence of an NF-AT gene. Such a diagnostic composition can be used in connection with a diagnostic method according to the invention. It may comprise further components commonly used in diagnosis, such as buffers.

With respect to the antibodies the same holds true which was already stated above in connection with the diagnostic method of the invention. The nucleic acid molecule contained in the diagnostic composition may, e.g., be a hybridization probe, a PCR primer and/or a nucleic acid molecule comprising an NF-AT binding site as already described in connection with the diagnostic method according to the invention.

The present invention also relates to a method for identifying a compound which increases NF-AT activity comprising the steps of
(a) incubating a compound to be tested for its ability to increase NF-AT activity with an NF-AT and a DNA construct containing an NF-AT binding site; and
(b) determining whether the presence of the compound leads to an increase of DNA binding of the NF-AT and/or of transcriptional activation conferred by the NF-AT.

The increase in DNA binding of the NF-AT may be determined by methods well known to the person skilled in the art. Examples are electrophoretic mobility shift assays, DNase I and other footprinting techniques, etc. Such a method may be carried out, e.g., by incubating in vitro an NF-AT together with the compound to be tested and a DNA molecule containing an NF-AT binding site. As a control the NF-AT is incubated with the DNA molecule in the absence of the compound. An increase of binding of NF-AT to its binding site in the DNA molecule in comparison to the control is indicative for an activation of the NF-AT by the compound.

The complexes that are formed with NF-AT alone or along with co-factors, e.g. AP-1, and DNA are enhanced by agents, such as ionomycin, i.e. an Ca++-ionophore. Thus, drug screening for identifying a compound which increases NF-AT activity may be performed with the DNA-protein complexes to determine if a candidate agent is capable to enhance the generation of NF-AT/DNA complexes. An assay of interest determines the ability of a candidate agent to mimic the effects of ionomycin in supporting NF-AT complexes. Such agents may then find use in chemotherapy against lymphomas and other neoplasias whose generation is facilitated by the lack of NF-AT expression or they may serve as lead compounds for the generation of more effective and non-toxic drugs.

The increase of transcriptional activation may be determined, e.g., by determining the amount of the synthesized RNA or protein of a sequence which is placed under the control of a promoter which can be activated by NF-AT.

Such a method can, for example, be carried out in vitro by using an in vitro transcription or transcription and translation system containing an NF-AT, the compound to be tested and a DNA construct containing an expression cassette. This expression cassette contains a promoter which is under NF-AT control (e.g. the interleukin 2, 4 or 5 promoters) and preferably a sequence linked thereto the expression of which can be easily detected with methods well known in the art. Examples for such a sequence are reporter genes such as the luciferase gene. As a control the NF-AT is incubated with the DNA construct in the absence of the compound. An increase in transcriptional activation by NF-AT in comparison to the control is indicative for the activation of NF-AT by the compound. Alternatively, such a method may also be carried out by transient transfection assays using NF-AT expression vectors and determining the level of transcriptional activation of a DNA sequence which is placed under the control of a promoter which can be activated by an NF-AT in the presence and in the absence of the compound to be tested.

The term “compound” or “agent” as used herein describes any molecule, e.g. protein or pharmaceutical, with the capability of altering, for example increasing, or mimicking the physiological function of a target protein. Preferably, the compound or agent has a low toxicity for human cells. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

The present invention also relates to a process for the preparation of a pharmaceutical composition comprising the step of formulating the compound identified by a method according to the invention as described above into a pharmaceutical composition.

The compounds having the desired pharmacological activity may be formulated as a pharmaceutical composition, e.g., by mixing with a pharmaceutically acceptable carrier and may be administered to a host for treatment of cancer, etc., or to otherwise enhance NF-AT function. The agents may be administered in a variety of ways, orally, topically, parenterally e.g. subcutaneously, intraperitoneally, by viral infection, intravascularly, etc. Topical treatments are of particular interest. Depending upon the manner of introduction, the compounds may be formulated in a variety of ways. The concentration of therapeutically active compound in the formulation may vary from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

The agents of the present invention can be used in native form or can be modified to form a chemical derivative. As used herein, a molecule is said to be a “chemical derivative” of another molecule when it contains additional chemical moieties not normally a part of the molecule. Such moieties may improve the molecule's solubility, absorption, biological half life, etc. The moieties may alternatively decrease the toxicity of the molecule, eliminate or attenuate any undesirable side effect of the molecule, etc. Moieties capable of present invention can be administered concurrently with, prior to, or following the administration of the other agent.

The agents or compounds of the present invention are administered to the mammal in a pharmaceutically acceptable form and in a therapeutically effective concentration. A composition is said to be “pharmacologically acceptable” if its administration can be tolerated by a recipient patient. Such an agent is said to be administered in a “therapeutically effective amount” if the amount administered is physiologically significant. An agent is physiologically significant if its presence results in a detectable change in the physiology of a recipient patient.

The agents of the present invention can be formulated according to known methods to prepare pharmaceutically useful compositions, whereby these materials, or their functional derivatives, are combined in admixture with a pharmaceutically acceptable carrier vehicle. Suitable vehicles and their formulation, inclusive of other human proteins, e.g., human serum albumin, are described, for example, in Remington's Pharmaceutical Sciences (16th ed., Osol, A., Ed., Mack, Easton Pa. (1980)). In order to form a pharmaceutically acceptable composition suitable for effective administration, such compositions will contain an effective amount of one or more of the agents of the present invention, together with a suitable amount of carrier vehicle. Additional pharmaceutical methods may be employed to control the duration of action. Control release preparations may be achieved through the use of polymers to complex or absorb one or more of the agents of the present invention. The controlled delivery may be exercised by selecting appropriate macromolecules (for example polyesters, polyamino acids, polyvinyl, pyrrolidone, ethylenevinylacetate, methylcellulose, carboxymethylcellulose, or protamine, sulfate) and the concentration of macromolecules as well as the methods of incorporation in order to control release. Another possible method to control the duration of action by controlled release preparations is to incorporate agents of the present invention into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, by coacervation techniques or by interfacial polymerization,for example, hydroxymethylcelluloseor gelatine microcapsules; and poly(methylmethacylate) microcapsules, respectively, or in colloidal drug delivery systems, for example, liposomes, albumin microspheres, microemulsions, nanoparticles, and nanocapsules or in macroemulsions. The following examples are offered by way of illustration and not by way of limitation.

The described pharmaceutical compositions can be used to prevent or treat neoplasias as defined above.

The present invention also relates to a pharmaceutical composition comprising an NF-AT protein and/or a nucleic acid molecule encoding an NF-AT protein optionally in combination with a pharmaceutically acceptable carrier. The NF-AT protein may be any NF-AT protein as described above. Furthermore, it may be a modified form of such an NF-AT protein or a fragment of such a protein which retains the ability to transactivate transcription in a manner similar to the naturally occurring NF-AT protein. As indicated above, the pharmaceutical composition may also comprise a nucleic acid molecule encoding an NF-AT. Such a nucleic acid molecule preferably also contains sequences which ensure transcription and translation of the NF-AT encoding sequence in the desired host cells. Such a pharmaceutical composition may be used for expressing the NF-AT protein in vivo, which is often referred to as gene therapy. In this context, the cells of a patient may, for example, be engineered ex vivo with a nucleic acid molecule encoding an NF-AT. The engineered cells may then be transferred to a patient in need of the NF-AT. Methods for engineering cells ex vivo with nucleic acid molecules are well known in the art and include, e.g., the use of retroviral particles containing RNA which encodes the NF-AT protein.

Alternatively, it is also possible to engineer cells in vivo by methods known in the art. Such methods include, e.g., the use of adenoviral vectors or producer cells which produce a retroviral particle containing RNA encoding the NF-AT protein. The pharmaceutical composition may be formulated as described in detail above.

In a preferred embodiment an NF-AT encoding nucleic acid molecule is used for a somatic hematopoietic stem cell (HSC) therapy.

The described pharmaceutical compositions can be used to prevent or treat neoplasias as defined above.

In case it contains NF-ATc1 or 2 or a corresponding nucleic acid molecule, the pharmaceutical composition is particulary useful to treat neoplasias based on hematopoietic cells, in particular lymphocytes, such as lymphoma.

In case the pharmaceutical composition comprises NF-ATc2 or a corresponding nucleic acid molecule, it is particularly useful for treating defects in chondrocytes/osteocytes.

Moreover, the present invention relates to a method for preventing or treating a neoplasia by increasing the activity of an NF-AT transcription factor.

The neoplasia can be a neoplasia as defined above. The increase of activity of an NF-AT transcription factor may be achieved, e.g., by administering to a patient in need thereof an effective amount of an NF-AT protein, of a nucleic acid molecule encoding an NF-AT protein or of a compound identified by a screening method according to the invention which increases the activity of an NF-AT or of a pharmaceutical composition as described above.

FIG. 1 shows schematically the structure of NF-AT factors

The DNA binding regions of NF-ATs, the RSD (‘Rel similarity domain’), are indicated. The N- and C-terminal transactivation domains, TAD-A and TAD-B, are also indicated. Further sequence motifs are shown for NF-ATc1 only where TAD-B consists of two peptides which are separated by an inhibitory domain. For the regulatory domain located between TAD-A and the RSD, the position of serine rich region, SRR, and of SP motifs 1-3 are indicated. In addition, binding regions for the transcriptional co-factor CBP and the Ca⁺⁺-dependent phosphatase calcineurin as well as signals for the nuclear localisation (NLS) and nuclear export (NES) of NF-ATc1 are shown.

FIG. 2 shows schematically the activation of NF-AT factors

T cell receptor (TCR) stimulation leads to a rapid activation of protein tyrosine kinases (PTKs), in particular of p56^{Ick}, and a rise of free, intracellular Ca⁺⁺. One downstream event of activation of PTKs is the generation of active, GTP-bound form of p21^{ras} which, in turn, activates downstream kinase cascades, such as the Raf-MEK-Erk cascade. A rise in intracellular free Ca⁺⁺ and calmodulin stimulates calcineurin (PP2B) activity which is crucially involved in the nuclear import and export of NF-AT factors. It is thought that calcineurin-mediated dephosphorylation of regulatory region (RR) of NF-AT factors unmasks nuclear localisation signals (NLS, see FIG. 1) and leads to their nuclear translocation. Both the Ser/Thr-specific protein kinases GSK, CKI, CKII and JNK have been described to phosphorylate NF-Atc1 and/or NF-Atc3, respectively, and to counteract calcineurin activity. These protein kinases and calcineurin appear to bind to NF-AT, to be translocated into the nucleus and to mediate the nuclear export of NF-AT. At numerous promoters active in T lymphocytes, NF-AT binds in synergy with AP-1 factors.

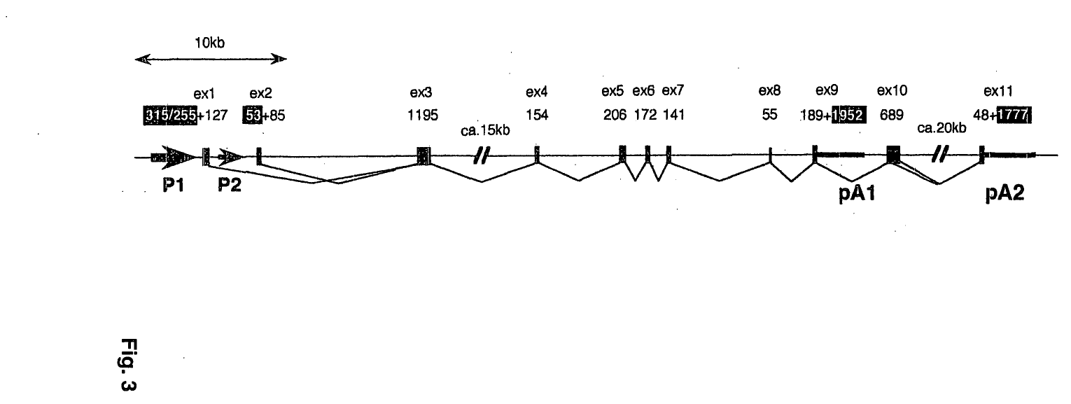

FIG. 3 shows the schematic structure of the murine chromosomal NF-ATc1 gene.

The lengths of 11 exons in bp is indicated above the gene. White numbers in black fields indicate the lengths of 5′ and 3′ untranslated mRNA regions, black numbers the lengths of protein coding segments. The positions of both promoters P1 and P2 (labelled by arrows) and of poly A sites pA1 and pA2 are indicated below. The entire gene was isolated by cloning of overlapping DNA fragments in BAC, cosmid and lambda vectors. All exons and large portions of introns have been sequenced.

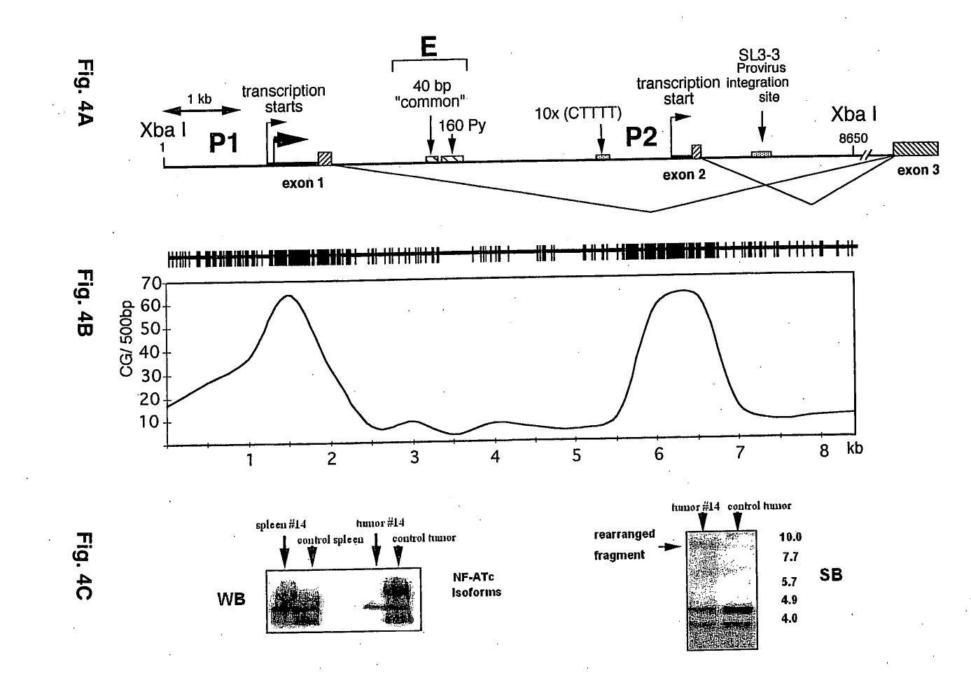

FIG. 4 shows the organisation of the NF-ATc1 promoter region. (A) Structure of the 8.65 bp Xba I DNA fragment harboring the two promoters P1 and P2. The protein coding portions of exons 1 and 2 are indicated by dashed boxes, their 5′ untranslated mRNA regions by thick black bars. Horizontal arrows before both exons indicate transcriptional start sites. E marks an intronic transcriptional enhancer which is able to increase transcription from P1. Sequence motifs within intron 1 are a stretch of 40 bp which is found in several other genes alternatively spliced at their 5′ ends, a stretch of 160 pyrimidines and 10 copies of sequence CTTTT. Note behind exon 2 an integration site for the retrovirus SL3-3, a potent inducer of T cell lymphomas (Sorensen et al., J. Virol. 70 (1996), 4063-70).

(B) Distribution of CpG residues over the promoter region. Above, one vertical dash indicates one CpG residue. The graph below shows the distribution of 375 bp CpGs within 500 bp intervals of Xba I fragment.

(C) Left: Immunoblot of whole cellular protein from spleens and tumors of mice. Animal #14 contained a proviral SL3-3 insertion within the NF-ATc1 promoter region, the control animal contained a proviral insertion in another gene. Note the suppression of NF-ATc1 expression in the T cell tumor from animal #14. Right: Detection of an additional 10 kb NF-ATc1 fragment in DNA from animal #14 indicating the clonal origin of SL3-3 proviral insertion.

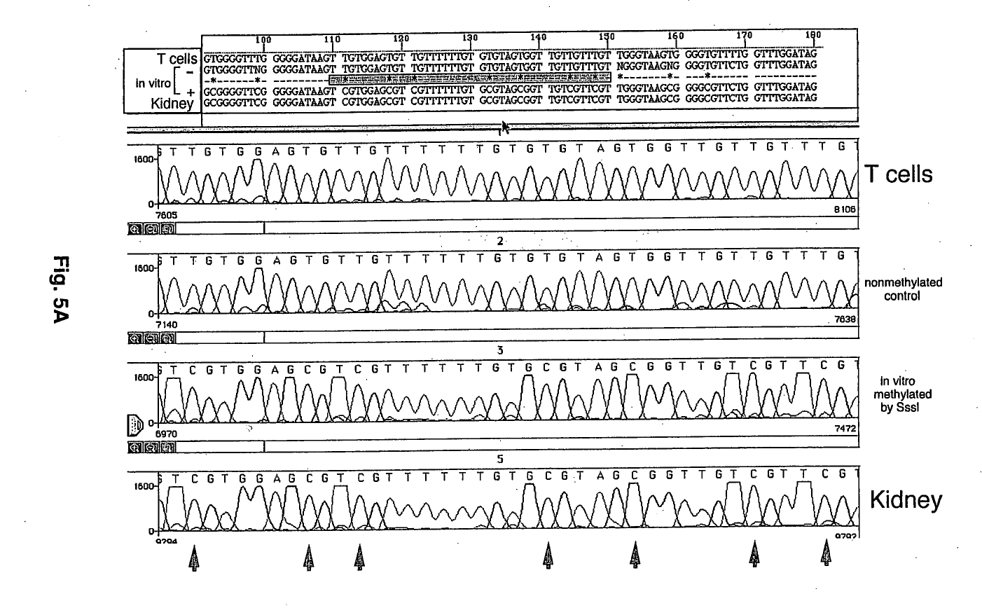

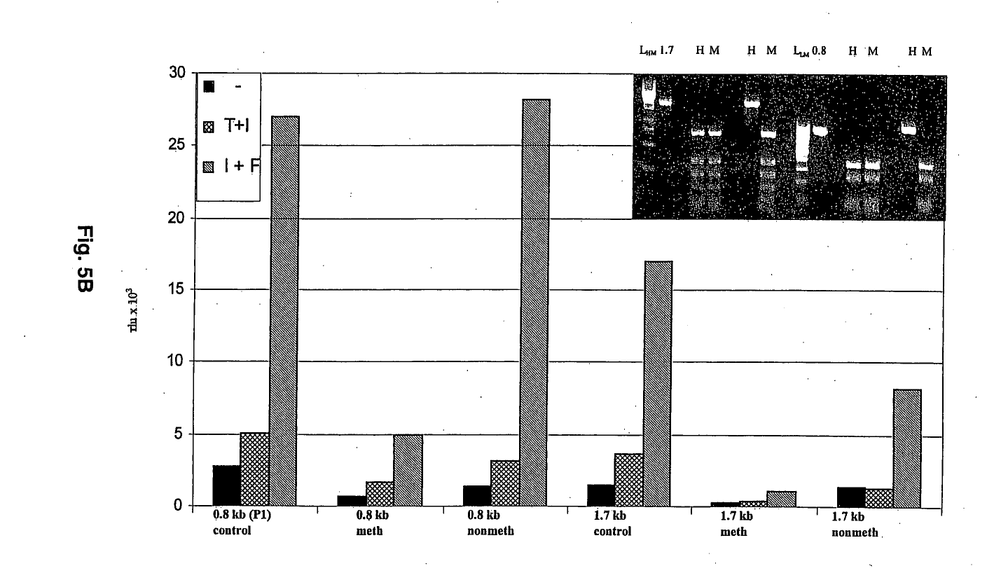

FIG. 5 shows that NF-ATc1 P1 promoter DNA is non-methylated in effector T cells but fully methylated in kidney cells.

(a) DNA Methylation studies. The DNA from murine T cells maintained after a primary CD3/CD28 stimulation for 5 days in vitro in the presence of IL-2 and from murine kidney was treated with Na-bisulfite according to Frommer et al., Proc. Natl. Acad. Sci. USA 89 (1992), 1827-183 and Raizis et al., Analyt. Biochem. 226 (1995), 161-166. In the same way P1 DNA amplified in bacteria which was either left unmethylated or fully methylated by use of DNA methylase Sss I was modified. In parallel the −1/−300 P1 DNA fragment was PCR amplified from the four DNA preparations using the DNA primers described in Materials and Methods. The PCR products were sequenced according to convential techniques using an automatic ABI 373 DNA sequencer. Shown is a selected sequence of four DNA samples from the central portion of the −1/−300 P1 fragment. The arrows below indicate the hypermethylation in kidney DNA and the non-methylated state of T cell DNA.

(b) DNA Methylation suppresses NF-ATc1 P1 promoter induction. P1 fragments of 0.8 kb (P1) or 1.7 kb in lenght were either left unmethylated or methylated in vitro using Sss I DNA methylase. The insert on the right hand indicates by Hpa II (H) and Msp I (M) cleavage that the promoter DNA was fully methylated (Hpa II is unable, Msp I is able to cleave methylated CCGG sites). After treatment the DNA fragments were cloned in front-of a luciferase gene, and the constructs were transfected into EL-4 cells using a conventional DEAE transfection protocol. One day later, the cells were either left untreated (-) or treated by TPA and ionomycin (T+I) or ionomycin and forskolin (I+F) for 24 hours. Shown are the relative luciferase units (rlu) of transfected cells.

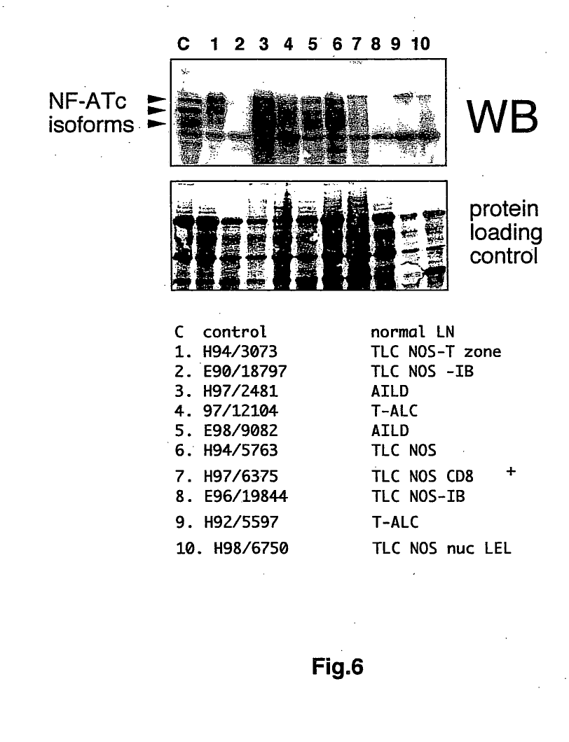

FIG. 6 shows suppression of NF-ATc1 expression in human T cell lymphomas.

Whole cell protein was prepared from human lymphnode cells (C; normal LN) or various samples from 10 T cells lymphomas and fractionated on a SDS-10% polyacrylamide gel. The protein staining of the gel is shown below as a “protein loading control”; the chemiluminescence of western blot (WB) obtained after incubation with an antibody raised against NF-ATc1/A (mAb 7A6: Northrop et al., Nature 369 (1994), 497-502) is shown on top.

The following examples serve to illustrate the invention.

### IN THE EXAMPLES THE FOLLOWING MATERIALS AND METHODS ARE USED

1. Cells and DNA Transfections

All lymphoid cells were grown to a density of 2×10⁵ cells/ml in RPMI medium containing 5% fetal calf serum. They were induced with TPA (20 ng/ml), ionomycin (0.5 μM) or TPA+ionomycin (T+I) as indicated. In transient transfections of murine El4 T lymphoma cells, 10 μg DNA was transfected into 2.5×10⁷ cells using a convential DEAE-dextran transfection protocol.

For the differentiation of naive CD4⁺CD62I^{hi} murine T cells in vitro, naive T cells were isolated from the spleens of BALB/cAnn mice using anti-CD4 and anti-CD62L Abs coupled to dynabeads. The cells were stimulated with anti-TCRβ (2.5 μg/ml) and anti-CD28 Abs (5 μg/ml) and incubated in Iscove's medium in the presence of either IL-12 (1000 U/ml), IL-2 (10 ng/ml) and anti-IL-4 Abs (10 μg/l) for Th1 or IL-4 (1000 U/ml) and anti-IFNγ Abs (10 μg/ml) for Th2 differentiation for 3 d followed by incubation for 4 d in the presence of IL-2 alone. The resulting Th1 and Th2 cells were stimulated for 5 h with T+I or anti-TCR α+β Abs.

2. Cloning and DNA Sequencing of the Murine Chromosomal NF-ATc1 Gene

The murine chromosomal NF-ATc1 gene was cloned by screening lambda phage, cosmid and BAC libraries using cDNAs of human NF-ATc1 isoforms (see Chuvpilo et al., Immunity 10 (1999) 261-269, and Chuvpilo et al., Immunity 16 (2002), 881-895). The corresponding clones were isolated, and their coding portions and large parts of the introns were sequened using an automatic ABI 373 sequencing apparatus according to convential protocols.

3. Immunoblots

Whole cellular protein extracts were prepared and immunoblot assays were done as described by Neumann et al. (EMBO J. 14 (1995), 1991-2004). For detection of proteins, appropriate peroxidase-coupled secondary antibodies were used with a standard enhanced chemiluminescence system (Amersham).

4. DNA Methylation Studies

CpG methylation of promoter DNA was investigated after DNA Na-bisulfite modification according to published protocols (Frommer et al., Proc. Natl. Acad. Sci. USA 89 (1992), 1827-1831; Raizis, et al., Analyt. Biochem. 226 (1995), 161-166). The following primers were used in PCR amplifications of modified genomic DNA and following DNA sequencing:

(SEQ ID NO:13)

310-asen dir
GTTTTGTTTTTTGTTTTTTAAAGTTGGAAAATATTTT

(SEQ ID NO:14)

310-asen dir nest
TTTTTTAAAGTTGGAAAATATTTTTTTYGGTTTT

(SEQ ID NO:15)

310-asen rev
ACCTTTACACACCTCTAAAAACTCCCTCCAATCCC

(SEQ ID NO:16)

310-asen rev nest
ACTCCCTCCAATCCCTTGTATCCTCATTACC

(SEQ ID NO:17)

310-sense dir
GTTTTTGTATATTTTTGGGAGTTTTTTTTAGTTTTTTG

(SEQ ID NO:18)

310-sense dir nest
TGGGAGTTTTTTTTAGTTTTTTGTGTTTTTATTAT

(SEQ ID NO:19)

310-sense rev
ACTCTACCTTCTACCTTTTTAAAACTAAAAAACACC

(SEQ ID NO:20)

310-sense rev nest
TTTTTAAAACTAAAAAACACCTCCCCCRACTC

### EXAMPLE 1

### Determination of the Structure of the Chromosomal Murine NF-ATc1 Gene, Particularly of its Promoter Region

The murine chromosomal NF-ATc1 gene located on chromosome 18 band E4 consists of 11 exons which are spread over more than 100 kb DNA (see FIG. 3) Similar to the human NF-ATc1 gene (see Serfling et al., Biochim. Biophys. Acta 1498 (2000), 1-18, for a review) the murine gene is expressed in several isoforms which differ both in their N- and C-terminal peptides. The short NF-ATc1/A isoform is predominantly expressed in T effector cells and lacks the C terminal peptide sequences specific for the isoforms B and C. These are encoded in exons 10 and 11 and share approximately 31% sequence homology with the C terminal peptide of murine NF-ATp (Chuvpilo et al., Immunity 10 (1999), 261-269). Similar to the generation of human NF-ATc1 isoforms the synthesis of murine NF-ATc1 isoforms is controlled by the activity of (at least) two poly A sites which are located behind exon 9 (pA1) and exon 11 (pA2), respectively (see FIG. 3 and Tyrsin et al., in prep.).

The transcription of both the human and murine NF-ATc1 gene results in two RNAs containing different 5′ ends. The majority of NF-ATc1 RNAs, designated as NF-ATc.α (Park et al., J. Biol. Chem. 271 (1996), 20914-10921), codes for an N terminal peptide of 42 amino acids which is missing in a minor RNA fraction. This minor fraction, NF-ATc.β, encodes a unique N terminal peptide of 29 or 27 amino acids in man or mouse, respectively (Park et al., J. Biol. Chem. 271 (1996), 20914-10921; Chuvpilo et al., Immunity 10 (1999), 261-269). These findings and the structure of NF-ATc1 5′ region suggest that NF-ATc1 RNAs are synthesized from two promoters and by alternate splicing events (see Serfling et al., Biochim. Biophys. Acta 1498 (2000), 1-18).

The 5′ region of murine NF-ATc1 gene (FIG. 4A) cloned in a Xba I fragment of 8650 bp contains the most distal exon 1 which encodes the 42 amino acids of NF-ATc.α, and exon 2 encoding the 27 amino acids in NF-ATc.β. Exons 1 and 2 are separated from each other by an intron of approximately 4 kb. Conspicous sequence properties are 375 CpG dinucleotides, i.e. potential targets for DNA methylation, which are clustered around the transcriptional start sites and within the 5′ untranslated mRNA regions (FIG. 4B). Sequence features within intron 1 are a stretch of 160 pyrimidines, 10 copies of the pentanucleotide CTTTT and a sequence of 40 bp that is 87.5% identical to a sequence within intron 1 of the murine whn nude gene. It is notable that similar to the NF-ATc1 gene the expression of whn that encodes a transcription factor of forkhead/winged helix family is controlled by two promoters and alternate splicing events. In addition, approximately 430 bp downstream from exon 2 an integration site was detected for the murine retrovirus SL3-3, a potent inducer of T cell lymphomas (Sorensen et al., J. Virol 70 (1996) 4063-4070). As documented by Southern blot hybridizations this is a clonal integration site affecting the majority of cell population (FIG. 4C). Moreover, the integration of provirus SL3-3 exerted a deleterious effect on NF-ATc1 expression in lymphoma tissue, i.e. it suppressed completely NF-ATc1 expression (FIG. 4C). This surprising finding suggests that—among other criteria—NF-ATc1 might act as a tumor suppressor for the generation of T cell lymphomas.

### EXAMPLE 2

### Methylation of NF-ATc1-P1 Promoter DNA

The distribution of CpG dinucleotides within the NF-ATc1 5′ region (FIG. 4B) suggests that DNA methylation plays an important role in the control of NF-ATc1 transcription in lymphoid cells. To correlate the methylation status of P1 promoter DNA with its activity, chromosomal DNA from various cell types was isolated and the methylation around the P1 promoter was investigated by sequencing genomic NF-ATc1 promoter DNA after modification with Na-bisulfite which converts unmethylated, but not methylated Cs to Ts (Frommer et al., Proc. Natl. Acad. Sci. USA 89 (1992), 1827-1831). As demonstrated in FIG. 5a for a short promoter fragment, the CpG residues within the P1 fragment from position 1- to -300 from the immediate promoter region were found to be methylated in genomic DNA isolated from embryonic stem cells and kidney cells in which NF-ATc1 is not expressed. In contrast, all these residues were non-methylated in genomic DNA isolated from Th1 and Th2 effector cells. A somewhat “intermediary” status was observed for DNA isolated from naive T lymphocytes isolated from murine lymphnodes. In this DNA several CpG residues were found to be methylated to approximately 50% suggesting that the expression of one of both NF-ATc1 alleles is controlled by methylation.

In order to show how DNA methylation might affect NF-ATc1 transcription P1 fragments of 0.8 kb and 1.7 kb were isolated and methylated in vitro using Sss I methylase. As shown in the insert of FIG. 5b by the cleavage of restriction enzymes Hpa II and Msp I (whose cleavage is either sensitive (Hpa II) or resistant against methylation (Msp I)) Sss I led to methylation of all CGCG recognition motifs within both DNA fragments. When these methylated promoter fragments were inserted in front of a luciferase gene and tested in EL-4 cells a five-fold decrease in the I+F-mediated induction of promoter activity was detected (FIG. 5b). This indicates that DNA methylation impairs P1 induction. A similar mechanism could also take in the inactivation of one NF-ATc1 allele during generation of T cell lymphomas.

### EXAMPLE 3

### NF-ATc1 Expression is Suppressed in a Large Part of Human T Cell Lymphomas

The suppression of NF-ATc1 expression in murine T cell lymphomas correlated with the integration of proviral SL3-3 DNA into the NF-ATc1 promoter region (FIGS. 4A and C) prompted the inventors to study the expression of a pannel of human T cell lymphomas which were provided by Dr. G. Ott (Pathol. Institut, Univ. Würzburg). When whole cellular protein extracts from these lymphomas were immunoblotted after fractionation on SDS-polyacrylamide gels by an antibody raised against NF-ATc1 one could observe that in about 50% of T cell lymphomas the expression of three NF-ATc1 isoforms A, B and C was switched off (FIG. 6). While in the protein extracts from normal lymph node cells and in 50% of the other T cell lymphoma samples the expression of these three NF-ATc1 isoforms was detected, apart from an unspecific band no indication for NF-ATc1 expression could be detected in 50% of T cell lymphomas. This is a further indication that NF-AT factors, in particular NF-ATc1, might act as a tumor suppressor for the generation of T cell lymphomas.

1. A diagnostic method comprising the step of determining in a sample taken from a patient the expression level and/or activity of one or more NF-AT transcription factor(s), and wherein a decrease or loss of the expression and/or activity of the NF-AT transcription factor(s) is indicative for the occurrence of a neoplasia.

2. The diagnostic method of claim 1, wherein the neoplasia is a neoplasia of hematopoietic cells, of chondrocytes, of osteocytes, of heart cells, muscle cells or blood vessel cells.

3. The diagnostic method of claim 1, wherein the neoplasia is a lymphoma.

4. The diagnostic method of claim 3, wherein the lymphoma is a T cell lymphoma a B-cell lymphoma, or Hodgkin lymphoma.

5. The diagnostic method of claim 1, wherein the sample is a biopsy.

6. The diagnostic method of claim 5, wherein the sample is a biopsy from a lymph node.

7. The diagnostic method of claim 1, wherein the sample is a blood sample.

8. The diagnostic method of claim 1, wherein the NF-AT transcription factor is selected from the group consisting of:
(a) NF-ATc1;
(b) NF-ATc2;
(c) NF-ATc3;
(d) NF-ATc4; and
a combination thereof.

9. The diagnostic method of claim 1, wherein the decrease or loss of the expression and/or activity of the NF-AT transcription factor(s) is detected by determining the amount of RNA encoding the NF-AT transcription factor(s) in the sample.

10. The diagnostic method of claim 1, wherein the decrease or loss of the expression and/or activity of the NF-AT transcription factor(s) is detected by determining the amount of the NF-AT transcription factor(s) in the sample.

11. The diagnostic method of claim 1, wherein the decrease or loss of the expression and/or activity of the NF-AT transcription factor(s) is detected by determining the DNA binding or the transactivation activity of the NF-AT transcription factor(s) in the sample.

12. The diagnostic method of claim 1, wherein the decrease or loss of the expression and/or activity of the NF-AT transcription factor is determined by analysing the methylation state of the promoter of the NF-AT gene(s) or by detecting a loss of heterozygosity.

13. A diagnostic composition comprising a mixture of antibodies which are specific for more than one NF-AT transcription factors or a nucleic acid molecule specifically hybridizing with more than one nucleotide sequence encoding an NF-AT transcription factor or with a promoter sequence of more than one NF-AT gene.

14. A pharmaceutical composition comprising a polypeptide which comprises the C-terminal peptide of 246 amino acids of the isoform C of an NF-ATc1 transcription factor or a nucleic acid molecule encoding such a polypeptide and optionally a pharmaceutically acceptable carrier.

15. Use A method of preventing or treating neoplasia comprising administration of a pharmaceutically effective amount of the pharmaceutical composition of claim 14.


SEQUENCE LISTING (20 sequences)

SEQ ID NO: 1 - DNA, 2793 bp, Homo sapiens
  CDS (1)..(2793)
atgacggggctggaggaccaggagttcgacttcgagttcctcttcgagtttaaccagcgcgacgagggcgccgccgcggccgccccagaacactatggctatgcatcctccaacgtcagccccgccctgccgctccccacggcgcactccaccctgccggccccgtgccacaaccttcagacctccacaccgggcatcatcccgccggcggatcacccctcggggtacggagcagctttggacggtgggcccgcgggctacttcctctcctccggccacaccaggcctgatggggcccctgccctggagagtcctcgcatcgagataacctcgtgcttgggcctgtaccacaacaataaccagtttttccacgatgtggaggtggaagacgtcctccctagctccaaacggtccccctccacggccacgctgagtctgcccagcctggaggcctacagagacccctcgtgcctgagcccggccagcagcctgtcctcccggagctgcaactcagaggcctcctcctacgagtccaactactcgtacccgtacgcgtccccccagacgtcgccatggcagtctccctgcgtgtctcccaagaccacggaccccgaggagggctttccccgcgggctgggggcctgcacactgctgggttccccgcggcactccccctccacctcgccccgcgccagcgtcactgaggagagctggctgggtgcccgctcctccagacccgcgtccccttgcaacaagaggaagtacagcctcaacggccggcagccgccctactcaccccaccactcgcccacgccgtccccgcacggctccccgcgggtcagcgtgaccgacgactcgtggttgggcaacaccacccagtacaccagctcggccatcgtggccgccatcaacgcgctgaccaccgacagcagcctggacctgggagatggcgtccctgtcaagtcccgcaagaccaccctggagcagccgccctcagtggcgctcaaggtggagcccgtcggggaggacctgggcagccccccgcccccggccgacttcgcgcccgaagactactcctctttccagcacatcaggaagggcggcttctgcgaccagtacctggcggtgccgcagcacccctaccagtgggcgaagcccaagcccctgtcccctacgtcctacatgagcccgaccctgcccgccctggactggcagctgccgtcccactcaggcccgtatgagcttcggattgaggtgcagcccaagtcccaccaccgagcccactacgagacggagggcagccggggggccgtgaaggcgtcggccggaggacaccccatcgtgcagctgcatggctacttggagaatgagccgctgatgctgcagcttttcattgggacggcggacgaccgcctgctgcgcccgcacgccttctaccaggtgcaccgcatcacagggaagaccgtgtccaccaccagccacgaggccatcctctccaacaccaaagtcctggagatcccactcctgccggagaacagcatgcgagccgtcattgactgtgccggaatcctgaaactcagaaactccgacattgaacttcggaaaggagagacggacatcgggaggaagaacacacgggtacggctggtgttccgcgttcacgtcccgcaacccagcggccgcacgctgtccctgcaggtggcctccaaccccatcgaatgctcccagcgctcagctcaggagctgcctctggtggagaagcagagcacggacagctatccggtcgtgggcgggaagaagatggtcctgtctggccacaacttcctgcaggactccaaggtcattttcgtggagaaagccccagatggccaccatgtctgggagatggaagcgaaaactgaccgggacctgtgcaagccgaattctctggtggttgagatcccgccgtttcggaatcagaggataaccagccccgttcacgtcagtttctacgtctgcaacgggaagagaaagcgaagccagtaccagcgtttcacctaccttcccgccaacgttccaattataaaaacagaacccactgatgattatgagcctgctccaacctgtggaccggtgagccaggggttaagtcctctcccaagaccatactacagccagcagctcgcgatgccacccgaccccagctcctgcctcgtggccggcttcccgccctgtccgcagagaagcaccctgatgccagcagcccctggcgtgagccccaagctccacgacctttctcccgctgcctacaccaagggcgttgccagcccgggccactgtcacctcggactcccgcagccggccggagaggcccccgccgtccaggacgtgcccaggccagtggccacgcaccccggctcgcccgggcagccacccccggccctgctgccacagcaggtgagtgcgcctccaagcagtagctgcccccctggtctcgaacactcgctctgccccagcagcccctctcctccactcccgcctgccacccaagagccgacctgcctgcagccctgcagcccagcgtgcccgcccgccacgggccgcccgcagcacctgccgtccacggtccgcagggacgagtctccgactgccgggccacggctgctgccagaggtgcatgaggacggtagtcctaatttggcccctattcctgtaacggtcaagcgagagcctgaagagttggaccagttgtacctggatgacgtaaatgaaataatacgaaatgacctctccagcacgagcacccactcctag

SEQ ID NO: 2 - protein, 930 aa, Homo sapiens
MTGLEDQEFDFEFLFEFNQRDEGAAAAAPEHYGYASSNVSPALPLPTAHSTLPAPCHNLQTSTPGIIPPADHPSGYGAALDGGPAGYFLSSGHTRPDGAPALESPRIEITSCLGLYHNNNQFFHDVEVEDVLPSSKRSPSTATLSLPSLEAYRDPSCLSPASSLSSRSCNSEASSYESNYSYPYASPQTSPWQSPCVSPKTTDPEEGFPRGLGACTLLGSPRHSPSTSPRASVTEESWLGARSSRPASPCNKRKYSLNGRQPPYSPHHSPTPSPHGSPRVSVTDDSWLGNTTQYTSSAIVAAINALTTDSSLDLGDGVPVKSRKTTLEQPPSVALKVEPVGEDLGSPPPPADFAPEDYSSFQHIRKGGFCDQYLAVPQHPYQWAKPKPLSPTSYMSPTLPALDWQLPSHSGPYELRIEVQPKSHHRAHYETEGSRGAVKASAGGHPIVQLHGYLENEPLMLQLFIGTADDRLLRPHAFYQVHRITGKTVSTTSHEAILSNTKVLEIPLLPENSMRAVIDCAGILKLRNSDIELRKGETDIGRKNTRVRLVFRVHVPQPSGRTLSLQVASNPIECSQRSAQELPLVEKQSTDSYPVVGGKKMVLSGHNFLQDSKVIFVEKAPDGHHVWEMEAKTDRDLCKPNSLVVEIPPFRNQRITSPVHVSFYVCNGKRKRSQYQRFTYLPANVPIIKTEPTDDYEPAPTCGPVSQGLSPLPRPYYSQQLAMPPDPSSCLVAGFPPCPQRSTLMPAAPGVSPKLHDLSPAAYTKGVASPGHCHLGLPQPAGEAPAVQDVPRPVATHPGSPGQPPPALLPQQVSAPPSSSCPPGLEHSLCPSSPSPPLPPATQEPTCLQPCSPACPPATGRPQHLPSTVRRDESPTAGPRLLPEVHEDGSPNLAPIPVTVKREPEELDQLYLDDVNEIIRNDLSSTSTHS

SEQ ID NO: 3 - DNA, 2766 bp, Homo sapiens
  CDS (1)..(2766)
atgaacgcccccgagcggcagccccaacccgacggcggggacgccccaggccacgagcctgggggcagcccccaagacgagcttgacttctccatcctcttcgactatgagtatttgaatccgaacgaagaagagccgaatgcacataaggtcgccagcccaccctccggacccgcataccccgatgatgtaatggactatggcctcaagccatacagcccccttgctagtctctctggcgagccccccggccgattcggagagccggatagggtagggccgcagaagtttctgagcgcggccaagccagcaggggcctcgggcctgagccctcggatcgagatcactccgtcccacgaactgatccaggcagtggggcccctccgcatgagagacgcgggcctcctggtggagcagcctcccctggccggggtggccgccagcccgaggttcaccctgcccgtgcccggcttcgagggctaccgcgagccgctttgcttgagccccgctagcagcggctcctctgccagcttcatttctgacaccttctccccctacacctcgccctgcgtctcgcccaataacggcgggcccgacgacctgtgtccgcagtttcaaaacatccctgctcattattcccccagaacctcgccaataatgtcacctcgaaccagcctcgccgaggacagctgcctgggccgccactcgcccgtgccccgtccggcctcccgctcctcatcgcctggtgccaagcggaggcattcgtgcgccgaggccttggttgccctgccgcccggagcctcaccccagcgctcccggagcccctcgccgcagccctcatctcacgtggcaccccaggaccacggctccccggctgggtacccccctgtggctggctctgccgtgatcatggatgccctgaacagcctcgccacggactcgccttgtgggatcccccccaagatgtggaagaccagccctgacccctcgccggtgtctgccgccccatccaaggccggcctgcctcgccacatctacccggccgtggagttcctggggccctgcgagcagggcgagaggagaaactcggctccagaatccatcctgctggttccgcccacttggcccaagccgctggtgcctgccattcccatctgcagcatcccagtgactgcatccctccctccacttgagtggccgctgtccagtcagtcaggctcttacgagctgcggatcgaggtgcagcccaagccacatcaccgggcccactatgagacagaaggcagccgaggggctgtcaaagctccaactggaggccaccctgtggttcagctccatggctacatggaaaacaagcctctgggacttcagatcttcattgggacagctgatgagcggatccttaagccgcacgccttctaccaggtgcaccgaatcacggggaaaactgtcaccaccaccagctatgagaagatagtgggcaacaccaaagtcctggagatccccttggagcccaaaaacaacatgagggcaaccatcgactgtgcggggatcttgaagcttagaaacgccgacattgagctgcggaaaggcgagacggacattggaagaaagaacacgcgggtgagactggttttccgagttcacatcccagagtccagtggcagaatcgtctctttacagactgcatctaaccccatcgagtgctcccagcgatctgctcacgagctgcccatggttgaaagacaagacacagacagctgcctggtctatggcggccagcaaatgatcctcacggggcagaactttacatccgagtccaaagttgtgtttactgagaagaccacagatggacagcaaatttgggagatggaagccacggtggataaggacaagagccagcccaacatgctttttgttgagatccctgaatatcggaacaagcatatccgcacacctgtaaaagtgaacttctacgtcatcaatgggaagagaaaacgaagtcagcctcagcactttacctaccacccagtcccagccatcaagacggagcccacggatgaatatgaccccactctgatctgcagccccacccatggaggcctggggagccagccttactacccccagcacccgatggtggccgagtccccctcctgcctcgtggccaccatggctccctgccagcagttccgcacggggctctcatcccctgacgcccgctaccagcaacagaacccagcggccgtactctaccagcggagcaagagcctgagccccagcctgctgggctatcagcagccggccctcatggccgccccgctgtcccttgcggacgctcaccgctctgtgctggtgcacgccggctcccagggccagagctcagccctgctccacccctctccgaccaaccagcaggcctcgcctgtgatccactactcacccaccaaccagcagctgcgctgcggaagccaccaggagttccagcacatcatgtactgcgagaatttcgcaccaggcaccaccagacctggcccgcccccggtcagtcaaggtcagaggctgagcccgggttcctaccccacagtcattcagcagcagaatgccacgagccaaagagccgccaaaaacggacccccggtcagtgaccaaaaggaagtattacctgcgggggtgaccattaaacaggagcagaacttggaccagacctacttggatgatgagctgatagacacacaccttagctggatacaaaacatattatga

SEQ ID NO: 4 - protein, 921 aa, Homo sapiens [sequence omitted]

SEQ ID NO: 5 - DNA, 3207 bp, Homo sapiens
  CDS (1)..(3207)
atgactactgcaaactgtggcgcccacgacgagctcgacttcaaactcgtctttggcgaggacggggcgccggcgccgccgcccccgggctcgcggcctgcagatcttgagccagatgattgtgcatccatttacatctttaatgtagatccacctccatctactttaaccacaccactttgcttaccacatcatggattaccgtctcactcttctgttttgtcaccatcgtttcagctccaaagtcacaaaaactatgaaggaacttgtgagattcctgaatctaaatatagcccattaggtggtcccaaaccctttgagtgcccaagtattcaaattacatctatctctcctaactgtcatcaagaattagatgcacatgaagatgacctacagataaatgacccagaacgggaatttttggaaaggccttctagagatcatctctatcttcctcttgagccatcctaccgggagtcttctcttagtcctagtcctgccagcagcatctcttctaggagttggttctctgatgcatcttcttgtgaatcgctttcacatatttatgatgatgtggactcagagttgaatgaagctgcagcccgatttacccttggatcccctctgacttctcctggtggctctccagggggctgccctggagaagaaacttggcatcaacagtatggacttggacactcattatcacccaggcaatctccttgccactctcctagatccagtgtcactgatgagaattggctgagccccaggccagcctcaggaccctcatcaaggcccacatccccctgtgggaaacggaggcactccagtgctgaagtttgttatgctgggtccctttcaccccatcactcacctgttccttcacctggtcactcccccaggggaagtgtgacagaagatacgtggctcaatgcttctgtccatggtgggtcaggccttggccctgcagtttttccatttcagtactgtgtagagactgacatccctctcaaaacaaggaaaacttctgaagatcaagctgccatactaccaggaaaattagagctgtgttcagatgaccaagggagtttatcaccagcccgggagacttcaatagatgatggccttggatctcagtatcctttaaagaaagattcatgtggtgatcagtttctttcagttccttcaccctttacctggagcaaaccaaagcctggccacacccctatatttcgcacatcttcattacctccactagactggcctttaccagctcattttggacaatgtgaactgaaaatagaagtgcaacctaaaactcatcatcgagcccattatgaaactgaaggtagccgaggggcagtaaaagcatctactgggggacatcctgttgtgaagctcctgggctataacgaaaagccaataaatctacaaatgtttattgggacagcagatgatcgatatttacgacctcatgcattttaccaggtgcatcgaatcactgggaagacagtcgctactgcaagccaagagataataattgccagtacaaaagttctggaaattccacttcttcctgaaaataatatgtcagccagtattgattgtgcaggtattttgaaactccgcaattcagatatagaacttcgaaaaggagaaactgatattggcagaaagaatactagagtacgacttgtgtttcgtgtacacatcccacagcccagtggaaaagtcctttctctgcagatagcctctatacccgttgagtgctcccagcggtctgctcaagaacttcctcatattgagaagtacagtatcaacagttgttctgtaaatggaggtcatgaaatggttgtgactggatctaattttcttccagaatccaaaatcatttttcttgaaaaaggacaagatggacgacctcagtgggaggtagaagggaagataatcagggaaaaatgtcaaggggctcacattgtccttgaagttcctccatatcataacccagcagttacagctgcagtgcaggtgcacttttatctttgcaatggcaagaggaaaaaaagccagtctcaacgttttacttatacaccagttttgatgaagcaagaacacagagaagagattgatttgtcttcagttccatctttgcctgtgcctcatcctgctcagacccagaggccttcctctgattcagggtgttcacatgacagtgtactgtcaggacagagaagtttgatttgctccatcccacaaacatatgcatccatggtgacctcatcccatctgccacagttgcagtgtagagatgagagtgttagtaaagaacagcatatgattccttctccaattgtacaccagccttttcaagtcacaccaacacctcctgtggggtcttcctatcagcctatgcaaactaatgttgtgtacaatggaccaacttgtcttcctattaatgctgcctctagtcaagaatttgattcagttttgtttcagcaggatgcaactctttctggtttagtgaatcttggctgtcaaccactgtcatccataccatttcattcttcaaattcaggctcaacaggacatctcttagcccatacacctcattctgtgcataccctgcctcatctgcaatcaatgggatatcattgttcaaatacaggacaaagatctctttcttctccagtggctgaccagattacaggtcagccttcgtctcagttacaacctattacatatggtccttcacattcagggtctgctacaacagcttccccagcagcttctcatcccttggctagttcaccgctttctgggccaccatctcctcagcttcagcctatgccttaccaatctcctagctcaggaactgcctcatcaccgtctccagccaccagaatgcattctggacagcactcaactcaagcacaaagtacgggccaggggggtctttctgcaccttcatccttaatatgtcacagtttgtgtgatccagcgtcatttccacctgatggggcaactgtgagcattaaacctgaaccagaagatcgagagcctaactttgcaaccattggtctgcaggacatcactttagatgatgaccaatttatatctgacttggaacaccagccatcaggttcagcagagaaatggcctaaccacagtgtgctctcatgtccagctcctttctggagaatctag

SEQ ID NO: 6 - protein, 1068 aa, Homo sapiens
MTTANCGAHDELDFKLVFGEDGAPAPPPPGSRPADLEPDDCASIYIFNVDPPPSTLTTPLCLPHHGLPSHSSVLSPSFQLQSHKNYEGTCEIPESKYSPLGGPKPFECPSIQITSISPNCHQELDAHEDDLQINDPEREFLERPSRDHLYLPLEPSYRESSLSPSPASSISSRSWFSDASSCESLSHIYDDVDSELNEAAARFTLGSPLTSPGGSPGGCPGEETWHQQYGLGHSLSPRQSPCHSPRSSVTDENWLSPRPASGPSSRPTSPCGKRRHSSAEVCYAGSLSPHHSPVPSPGHSPRGSVTEDTWLNASVHGGSGLGPAVFPFQYCVETDIPLKTRKTSEDQAAILPGKLELCSDDQGSLSPARETSIDDGLGSQYPLKKDSCGDQFLSVPSPFTWSKPKPGHTPIFRTSSLPPLDWPLPAHFGQCELKIEVQPKTHHRAHYETEGSRGAVKASTGGHPVVKLLGYNEKPINLQMFIGTADDRYLRPHAFYQVHRITGKTVATASQEIIIASTKVLEIPLLPENNMSASIDCAGILKLRNSDIELRKGETDIGRKNTRVRLVFRVHIPQPSGKVLSLQIASIPVECSQRSAQELPHIEKYSINSCSVNGGHEMVVTGSNFLPESKIIFLEKGQDGRPQWEVEGKIIREKCQGAHIVLEVPPYHNPAVTAAVQVHFYLCNGKRKKSQSQRFTYTPVLMKQEHREEIDLSSVPSLPVPHPAQTQRPSSDSGCSHDSVLSGQRSLICSIPQTYASMVTSSHLPQLQCRDESVSKEQHMIPSPIVHQPFQVTPTPPVGSSYQPMQTNVVYNGPTCLPINAASSQEFDSVLFQQDATLSGLVNLGCQPLSSIPFHSSNSGSTGHLLAHTPHSVHTLPHLQSMGYHCSNTGQRSLSSPVADQITGQPSSQLQPITYGPSHSGSATTASPAASHPLASSPLSGPPSPQLQPMPYQSPSSGTASSPSPATRMHSGQHSTQAQSTGQGGLSAPSSLICHSLCDPASFPPDGATVSIKPEPEDREPNFATIGLQDITLDDDQFISDLEHQPSGSAEKWPNHSVLSCPAPFWRI

SEQ ID NO: 7 - DNA, 2709 bp, Homo sapiens
  CDS (1)..(2709)
atgggggcggccagctgcgaggatgaggagctggaatttaagctggtgttcggggaggaaaaggaggcccccccgctgggcgcggggggattgggggaagaactggactcagaggatgccccgccatgctgccgtctggccttgggagagccccctccctatggcgctgcacctatcggtattccccgacctccaccccctcggcctggcatgcattcgccaccgccgcgaccagccccctcacctggcacctgggagagccagcccgccaggtcggtgaggctgggaggaccaggagggggtgctgggggtgctgggggtggccgtgttctcgagtgtcccagcatccgcatcacctccatctctcccacgccggagccgccagcagcgctggaggacaaccctgatgcctggggggacggctctcctagagattaccccccaccagaaggctttgggggctacagagaagcaggggcccagggtgggggggccttcttcagcccaagccctggcagcagcagcctgtcctcgtggagcttcttctccgatgcctctgacgaggcagccctgtatgcagcctgcgacgaggtggagtctgagctaaatgaggcggcctcccgctttggcctgggctccccgctgccctcgccccgggcctcccctcggccatggacccccgaagatccctggagcctgtatggtccaagccccggaggccgagggccagaggatagctggctactcctcagtgctcctgggcccaccccagcctccccgcggcctgcctctccatgtggcaagcggcgctattccagctcgggaaccccatcttcagcctccccagctctgtcccgccgtggcagcctgggggaagaggggtctgagccacctccaccacccccattgcctctggcccgggacccgggctcccctggtccctttgactatgtgggggccccaccagctgagagcatccctcagaagacacggcggacttccagcgagcaggcagtggctctgcctcggtctgaggagcctgcctcatgcaatgggaagctgcccttgggagcagaggagtctgtggctcctccaggaggttcccggaaggaggtggctggcatggactacctggcagtgccctccccactcgcttggtccaaggcccggattgggggacacagccctatcttcaggacctctgccctacccccactggactggcctctgcccagccaatatgagcagctggagctgaggatcgaggtacagcctagagcccaccaccgggcccactatgagacagaaggcagccgtggagctgtcaaagctgcccctggcggtcaccccgtagtcaagctcctaggctacagtgagaagccactgaccctacagatgttcatcggcactgcagatgaaaggaacctgcggcctcatgccttctatcaggtgcaccgtatcacaggcaagatggtggccacggccagctatgaagccgtagtcagtggcaccaaggtgttggagatgactctgctgcctgagaacaacatggcggccaacattgactgcgcgggaatcctgaagcttcggaattcagacattgagcttcggaagggtgagacggacatcgggcgcaaaaacacacgtgtacggctggtgttccgggtacacgtgccccagggcggcgggaaggtcgtctcagtacaggcagcatcggtgcccatcgagtgctcccagcgctcagcccaggagctgccccaggtggaggcctacagccccagtgcctgctctgtgagaggaggcgaggaactggtactgaccggctccaacttcctgccagactccaaggtggtgttcattgagaggggtcctgatgggaagctgcaatgggaggaggaggccacagtgaaccgactgcagagcaacgaggtgacgctgaccctgactgtccccgagtacagcaacaagagggtttcccggccagtccaggtctacttttatgtctccaatgggcggaggaaacgcagtcctacccagagtttcaggtttctgcctgtgatctgcaaagaggagcccctaccggactcatctctgcggggtttcccttcagcatcggcaaccccctttggcactgacatggacttctcaccacccaggcccccctacccctcctatccccatgaagaccctgcttgcgaaactccttacctatcagaaggcttcggctatggcatgccccctctgtacccccagacggggcccccaccatcctacagaccgggcctgcggatgttccctgagactaggggtaccacaggttgtgcccaaccacctgcagtttccttccttccccgccccttccctagtgacccgtatggagggcggggctcctctttccccctggggctgccattctctccgccagccccctttcggccgcctcctcttcctgcatccccaccgcttgaaggccccttcccttcccagagtgatgtgcatcccctacctgctgagggatacaataaggtagggccaggctatggccctggggagggggctccggagcaggagaaatccaggggtggctacagcagcggctttcgagacagtgtccctatccagggtatcacgctggaggaagtgagtgagatcattggccgagacctgagtggcttccctgcacctcctggagaagagcctcctgcctga

SEQ ID NO: 8 - protein, 902 aa, Homo sapiens [sequence omitted]

SEQ ID NO: 9 - DNA, 2129 bp, Homo sapiens
  CDS (1)..(2127)
atgccaagcaccagctttccagtcccttccaagtttccacttggccctgcggctgcggtcttcgggagaggagaaactttggggcccgcgccgcgcgccggcggcaccatgaagtcagcggaggaagaacactatggctatgcatcctccaacgtcagccccgccctgccgctccccacggcgcactccaccctgccggccccgtgccacaaccttcagacctccacaccgggcatcatcccgccggcggatcacccctcggggtacggagcagctttggacggtgggcccgcgggctacttcctctcctccggccacaccaggcctgatggggcccctgccctggagagtcctcgcatcgagataacctcgtgcttgggcctgtaccacaacaataaccagtttttccacgatgtggaggtggaagacgtcctccctagctccaaacggtccccctccacggccacgctgagtctgcccagcctggaggcctacagagacccctcgtgcctgagcccggccagcagcctgtcctcccggagctgcaactcagaggcctcctcctacgagtccaactactcgtacccgtacgcgtccccccagacgtcgccatggcagtctccctgcgtgtctcccaagaccacggaccccgaggagggctttccccgcgggctgggggcctgcacactgctgggttccccgcggcactccccctccacctcgccccgcgccagcgtcactgaggagagctggctgggtgcccgctcctccagacccgcgtccccttgcaacaagaggaagtacagcctcaacggccggcagccgccctactcaccccaccactcgcccacgccgtccccgcacggctccccgcgggtcagcgtgaccgacgactcgtggttgggcaacaccacccagtacaccagctcggccatcgtggccgccatcaacgcgctgaccaccgacagcagcctggacctgggagatggcgtccctgtcaagtcccgcaagaccaccctggagcagccgccctcagtggcgctcaaggtggagcccgtcggggaggacctgggcagccccccgcccccggccgacttcgcgcccgaagactactcctctttccagcacatcaggaagggcggcttctgcgaccagtacctggcggtgccgcagcacccctaccagtgggcgaagcccaagcccctgtcccctacgtcctacatgagcccgaccctgcccgccctggactggcagctgccgtcccactcaggcccgtatgagcttcggattgaggtgcagcccaagtcccaccaccgagcccactacgagacggagggcagccggggggccgtgaaggcgtcggccggaggacaccccatcgtgcagctgcatggctacttggagaatgagccgctgatgctgcagcttttcattgggacggcggacgaccgcctgctgcgcccgcacgccttctaccaggtgcaccgcatcacagggaagaccgtgtccaccaccagccacgaggccatcctctccaacaccaaagtcctggagatcccactcctgccggagaacagcatgcgagccgtcattgactgtgccggaatcctgaaactcagaaactccgacattgaacttcggaaaggagagacggacatcgggaggaagaacacacgggtacggctggtgttccgcgttcacgtcccgcaacccagcggccgcacgctgtccctgcaggtggcctccaaccccatcgaatgctcccagcgctcagctcaggagctgcctctggtggagaagcagagcacggacagctatccggtcgtgggcgggaagaagatggtcctgtctggccacaacttcctgcaggactccaaggtcattttcgtggagaaagccccagatggccaccatgtctgggagatggaagcgaaaactgaccgggacctgtgcaagccgaattctctggtggttgagatcccgccgtttcggaatcagaggataaccagccccgttcacgtcagtttctacgtctgcaacgggaagagaaagcgaagccagtaccagcgtttcacctaccttcccgccaacggtaacgccatctttctaaccgtaagccgtgaacatga

SEQ ID NO: 10 - protein, 709 aa, Homo sapiens
MPSTSFPVPSKFPLGPAAAVFGRGETLGPAPRAGGTMKSAEEEHYGYASSNVSPALPLPTAHSTLPAPCHNLQTSTPGIIPPADHPSGYGAALDGGPAGYFLSSGHTRPDGAPALESPRIEITSCLGLYHNNNQFFHDVEVEDVLPSSKRSPSTATLSLPSLEAYRDPSCLSPASSLSSRSCNSEASSYESNYSYPYASPQTSPWQSPCVSPKTTDPEEGFPRGLGACTLLGSPRHSPSTSPRASVTEESWLGARSSRPASPCNKRKYSLNGRQPPYSPHHSPTPSPHGSPRVSVTDDSWLGNTTQYTSSAIVAAINALTTDSSLDLGDGVPVKSRKTTLEQPPSVALKVEPVGEDLGSPPPPADFAPEDYSSFQHIRKGGFCDQYLAVPQHPYQWAKPKPLSPTSYMSPTLPALDWQLPSHSGPYELRIEVQPKSHHRAHYETEGSRGAVKASAGGHPIVQLHGYLENEPLMLQLFIGTADDRLLRPHAFYQVHRITGKTVSTTSHEAILSNTKVLEIPLLPENSMRAVIDCAGILKLRNSDIELRKGETDIGRKNTRVRLVFRVHVPQPSGRTLSLQVASNPIECSQRSAQELPLVEKQSTDSYPVVGGKKMVLSGHNFLQDSKVIFVEKAPDGHHVWEMEAKTDRDLCKPNSLVVEIPPFRNQRITSPVHVSFYVCNGKRKRSQYQRFTYLPANGNAIFLTVSREH

SEQ ID NO: 11 - DNA, 23 bp, Artificial Sequence
  Description of Artificial Sequence artificial sequence
caycaycgrgcccaytaygarac

SEQ ID NO: 12 - DNA, 21 bp, Artificial Sequence
  Description of Artificial Sequence artificial sequence
tcbtgrgcwgabcgctgggag

SEQ ID NO: 13 - DNA, 38 bp, Artificial Sequence
  Description of Artificial Sequence artificial sequence
gttttgttttttgtttttttaaagttggaaaatatttt

SEQ ID NO: 14 - DNA, 35 bp, Artificial Sequence
  Description of Artificial Sequence artificial sequence
acctttacacacctctaaaaactccctccaatccc

SEQ ID NO: 15 - DNA, 34 bp, Artificial Sequence
  Description of Artificial Sequence artificial sequence
ttttttaaagttggaaaatatttttttyggtttt

SEQ ID NO: 16 - DNA, 31 bp, Artificial Sequence
  Description of Artificial Sequence artificial sequence
actccctccaatcccttgtatcctcattacc

SEQ ID NO: 17 - DNA, 38 bp, Artificial Sequence
  Description of Artificial Sequence artificial sequence
gtttttgtatatttttgggagttttttttagttttttg

SEQ ID NO: 18 - DNA, 35 bp, Artificial Sequence
  Description of Artificial Sequence artificial sequence
tgggagttttttttagttttttgtgtttttattat

SEQ ID NO: 19 - DNA, 36 bp, Artificial Sequence
  Description of Artificial Sequence artificial sequence
actctaccttctacctttttaaaactaaaaaacacc

SEQ ID NO: 20 - DNA, 32 bp, Artificial Sequence
  Description of Artificial Sequence artificial sequence
tttttaaaactaaaaaacacctcccccractc